(19) United States
(12) Patent Application Publication
(10) Pub. No.: US 2017/0356918 A1
(43) Pub. Date: Dec. 14, 2017

(54) MULTIPLEXED IMMUNOHISTOCHEMISTRY ASSAYS FOR DIAGNOSIS AND TREATMENT OF CANCER

(71) Applicant: IGNYTA, INC., San Diego, CA (US)

(72) Inventors: Jason Hans CHRISTIANSEN, Carlsbad, CA (US); Danielle Anthea MURPHY, Solana Beach, CA (US); Aaron Scott BOOMER, Vista, CA (US); Jennifer Lyn LAMOUREUX, Oceanside, CA (US)

(73) Assignee: IGNYTA, INC., San Diego, CA (US)

(21) Appl. No.: 15/533,000

(22) PCT Filed: Dec. 1, 2015
(86) PCT No.: PCT/US15/63163

§ 371 Date: Jun. 2, 2017

Related U.S. Application Data
(60) Provisional application No. 62/086,921, filed on Dec. 3, 2014.

(51) Int. Cl. G01N 33/574; G01N 27/447; A61K 31/496; C12Q 1/68

(52) U.S. Cl. CPC G01N 33/57484; C12Q 1/6813; G01N 33/57423; G01N 33/57411; G01N 33/57415; G01N 27/447; G01N 33/57438; G01N 33/57449; C12Q 1/686; A61K 31/496; G01N 2800/52; G01N 2800/7028; G01N 2033/57461

(57) ABSTRACT

The present disclosure generally relates to methods and compositions for identifying and/or treating cancer patients harboring one or more molecular alterations in clinically important biomarkers, preferably in multiplexed assays, such that multiple biomarkers can be assayed simultaneously. In one embodiment, the disclosure relates to methods for rapid screening large populations of biological samples by using a high-throughput multiplexed assay to assess relative prevalence of multiple indications, optionally followed by a second analytical assay with higher sensitivity and specificity.

### RELATED APPLICATIONS

The present application claims priority to U.S. Provisional Patent Application Ser. No. 62/086,921, filed on Dec. 3, 2014, the content of which is hereby expressly incorporated by reference in its entirety.

### FIELD

The present disclosure generally relates to methods and compositions for identifying and/or treating cancer patients harboring one or more molecular alterations in clinically important biomarkers, preferably in multiplexed assays, such that multiple biomarkers can be assayed simultaneously. In one embodiment, the disclosure relates to methods for rapid screening large populations of biological samples by using a high-throughput multiplexed assay to assess relative prevalence of multiple indications, optionally followed by a second analytical assay with higher sensitivity and specificity.

### BACKGROUND

Unless otherwise indicated herein, the materials described in this section are not prior art to the claims in this application and are not admitted to be prior art by inclusion in this section.

Cancer represents the phenotypic end-point of multiple genetic alterations that endow cells with a full range of biological properties required for tumorigenesis. Indeed, a hallmark genomic feature of many cancers, including, for example, lung cancer, breast cancer, ovarian cancer, pancreatic cancer, and colon cancer, is the presence of numerous complex chromosome structural aberrations and gene rearrangements, including translocations, intra-chromosomal inversions, point mutations, germline mutations, deletions, gene copy number changes, and gene expression level changes, among others.

There exists an ongoing need for methods for effectively and rapidly identifying one or more molecular alterations associated with cancer. Although a large number of cancer treatments presently exist, a number of additional potential cancer therapies (including those in the field of precision medicine that target uncommon one or more molecular alterations of cancer biomarkers) have been developed in the past decades. The challenge of identifying rare molecular variations in large patient populations can be time consuming and costly, especially when using highly sensitive methods. In addition, care providers are faced with the challenge of identifying small patient populations through the use of sensitive, reproducible testing or screening while maintaining testing efficiency and manageable costs.

### SUMMARY

In one aspect, disclosed herein are methods for treating cancer in a patient, including (a) acquiring knowledge of the presence of one or more molecular alterations in a biological sample from the cancer patient, wherein the one or more molecular alterations is detected by an assay comprising one or more antibodies that bind to one or more of ALK, ROS1, TrkA, TrkB, and TrkC biomarkers; (b) selecting a chemotherapeutic agent as a treatment for the cancer patient wherein the assay detects the presence of one or more of molecular alterations, and wherein the selected chemotherapeutic agent is one or more of N-[5-(3,5-difluorobenzyl)-1H-indazol-3-yl]-4-(piperazin-1-yl)-2-(tetrahydro-2H-pyran-4-ylamino) benzamide, N-[5-(3,5-difluorobenzyl)-1H-indazol-3-yl]-4-(4-methyl-piperazin-1-yl)-2-(tetrahydro-2H-pyran-4-ylamino) benzamide, and N-[5-(3,5-difluoro-benzenesulfonyl)-1H-indazol-3-yl]-2-((R)-2-methoxy-1-methyl-ethylamino)-4-(4-methyl-piperazin-1-yl) benzamide, or a pharmaceutically acceptable salt thereof; and (c) administering a therapeutically effective amount of the one or more selected chemotherapeutic agents to the cancer patient.

In another aspect, disclosed herein are methods for selecting a cancer patient who is predicted to respond to the administration of a therapeutic regimen, including (a) acquiring knowledge of the presence of one or more molecular alterations in a biological sample from the cancer patient, wherein the one or more molecular alterations is detected by an assay comprising one or more antibodies that bind to one or more of ALK, ROS1, TrkA, TrkB, and TrkC biomarkers; and (b) selecting the patient as predicted to respond to the administration of a therapeutic regimen if the one or more molecular alterations is detected in one or more of the biomarkers, or selecting the patient as predicted to not respond to the administration of a therapeutic regimen if the one or more molecular alterations is not detected in the biomarkers. In the methods according to this aspect of the disclosure, the therapeutic regiment includes administering to the selected patient a therapeutically effective amount of one or more chemotherapeutic agents selected from N-[5-(3,5-difluorobenzyl)-1H-indazol-3-yl]-4-(piperazin-1-yl)-2-(tetrahydro-2H-pyran-4-ylamino) benzamide, N-[5-(3,5-difluorobenzyl)-1H-indazol-3-yl]-4-(4-methyl-piperazin-1-yl)-2-(tetrahydro-2H-pyran-4-ylamino) benzamide, and N-[5-(3,5-difluoro-benzenesulfonyl)-1H-indazol-3-yl]-2-((R)-2-methoxy-1-methyl-ethylamino)-4-(4-methyl-piperazin-1-yl) benzamide, or a pharmaceutically acceptable salt thereof.

Implementations of the methods of the present disclosure can include one or more of the following features. In some embodiments, the selected chemotherapeutic agent is N-[5-(3,5-difluorobenzyl)-1H-indazol-3-yl]-4-(piperazin-1-yl)-2-(tetrahydro-2H-pyran-4-ylamino) benzamide, or a pharmaceutically acceptable salt thereof. In some embodiments, the selected chemotherapeutic agent is N-[5-(3,5-difluorobenzyl)-1H-indazol-3-yl]-4-(4-methyl-piperazin-1-yl)-2-(tetrahydro-2H-pyran-4-ylamino) benzamide, or a pharmaceutically acceptable salt thereof. In some embodiments, the selected chemotherapeutic agent is N-[5-(3,5-difluoro-benzenesulfonyl)-1H-indazol-3-yl]-2-((R)-2-methoxy-1-methyl-ethylamino)-4-(4-methyl-piperazin-1-yl) benzamide, or a pharmaceutically acceptable thereof. In some embodiments, the assay includes one or more antibodies that bind to at least two of ALK, ROS1, TrkA, TrkB and TrkC biomarkers. In some embodiments, the one or more molecular alterations detected in the biological sample involve at least two, at least three, or at least four of the biomarkers. In some embodiments, the knowledge of the presence of the one or more molecular alterations in the biological sample is acquired from an assay that includes contacting the biological sample with one or more antibodies or fragments thereof specific for the biomarkers. In some embodiments, the specific antibodies are monoclonal antibodies. In some embodiments, the specific antibodies include at least one of D5F3®, D4D5®, C17F1®, and combinations thereof. In some embodiments, the biological sample is contacted with one or more of the specific antibodies simultaneously. In some embodiments, the biological sample is sequentially contacted with the specific antibodies. In some embodiments, the one or more molecular alterations results in elevated expression of one or more of the ALK, ROS1, TrkA, TrkB, and TrkC biomarkers. In some embodiments, the knowledge of the one or more molecular alterations is acquired from an assay wherein determining whether the expression of one or more biomarker is elevated includes: (a) determining the expression level of the one or more biomarkers in the biological sample; and (b) comparing the determined expression level to a reference expression level. In some embodiments, the knowledge of the one or more molecular alterations is acquired from an antibody-based assay. In some embodiments, the antibody-based assay is selected from the group consisting of ELISA, immunohistochemistry, western blotting, mass spectrometry, flow cytometry, protein-microarray, immunofluorescence, and a multiplex detection assay. In some embodiments, the antibody-based assay includes an immunohistochemistry analysis.

In some embodiments, implementations of the methods disclosed herein further include acquiring knowledge of a genetic alteration in the cancer of the patient from a second analytical assay prior to the administering step, wherein the second analytical assay is selected from the group consisting of capillary electrophoresis, nucleic acid sequencing, polypeptide sequencing, restriction digestion, nucleic acid amplification-based assays, nucleic acid hybridization assay, comparative genomic hybridization, real-time PCR, quantitative reverse transcription PCR (qRT-PCR), PCR-RFLP assay, HPLC, mass-spectrometric genotyping, fluorescent in-situ hybridization (FISH), next generation sequencing (NGS), and a kinase activity assay. In some embodiments, the cancer is cancer is selected from the group consisting of anaplastic large-cell lymphoma (ALCL), colorectal cancer (CRC), cholangiocarcinoma, gastric, glioblastomas (GBM), leiomyosarcoma, melanoma, non-small cell lung cancer (NSCLC), squamous cell lung cancer, neuroblastoma (NB), ovarian cancer, pancreatic cancer, prostate cancer, medullary thyroid cancer, breast cancer, and papillary thyroid cancer. In some embodiments, the knowledge of the one or more molecular alterations is obtained from an assay performed simultaneously on a plurality of biological samples. In some embodiments, the plurality of biological samples includes at least 6, 12, 24, 48, 96, 200, 384, 400, 500, 1000, 1500, or 3000 samples. In some embodiments, the one or more molecular alterations is selected from a genetic mutation, a gene amplification, a gene rearrangement, a single-nucleotide variation (SNV), a deletion, an insertion, an InDel mutation, a single nucleotide point mutation (SNP), an epigenetic alteration, a splicing variant, an RNA/protein overexpression, an aberrant RNA/protein expression, and any combination thereof. In some embodiments, the one or more molecular alterations include an insertion of a heterologous nucleic acid sequence within a coding sequence of a biomarker gene. In some embodiments, the insertion forms a chimeric nucleic acid sequence that encodes a fusion peptide. In some embodiments, the acquiring knowledge of the one or more molecular alterations further includes determining a nucleic acid sequence and/or an amino acid sequence comprising the one or more molecular alterations.

In some embodiments, the selected chemotherapeutic agent or a pharmaceutically acceptable salt thereof is administered as a single therapeutic agent or in combination with a second therapeutic agent. In some embodiments, the methods disclosed herein include administering to the patient a therapeutically effective amount of the selected chemotherapeutic agent, or a pharmaceutically accepted salt thereof, in multiple dosages for a treatment period of 2 to 50 days. In some embodiments, the selected chemotherapeutic agent or a pharmaceutically acceptable salt thereof is administered to the patient in multiple dosages of about 50 to about 200 mg/kg per dose over a treatment period of 5 to 42 day. In some embodiments, the selected chemotherapeutic agent or a pharmaceutically acceptable salt thereof is administered to the patient with an oral dosage of about 60 mg/kg twice a day (BID), seven times per week. In some embodiments, the selected chemotherapeutic agent or a pharmaceutically acceptable salt thereof is administered to the patient with an oral dosage of about 60 mg/kg twice a day (BID), seven times per week for six weeks, on alternate weekly basis (i.e. one week on one week off).

The foregoing summary is illustrative only and is not intended to be in any way limiting. In addition to the illustrative aspects, alternatives, and features described above, further aspects, alternatives, objects and features of the disclosure will become fully apparent from the drawings and the following detailed description and the claims.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several alternatives in accordance with the disclosure and are not to be considered limiting of its scope.

FIG. 1 illustrates the results of multiplexed immunohistochemistry (IHC) experiments demonstrating the performance of a multiplexed IHC assay performed in accordance with some embodiments disclosed herein. The IHC assay was performed with an a mixture of antibodies, D5F3®, D4D5®, C17F1®, as described in Example 1. The following five control cell lines were used: Karpas299 (ALK+, TrkA overexpression); HCC78 (ROS1+); KM12 (ALK+, TrkA+); BaF3/NTRK2:ETV6 (TrkB+); and BaF3/NTRK3:ETV6 (TrkC+).

FIGS. 2A to 2C summarize the results of immunohistochemistry experiments performed as described in Example 1, to assess specificity of the monoclonal antibodies D5F3®, D4D5®, C17F1®, individually. The following five control cell lines were used: Karpas299 (ALK+, TrkA overexpression); HCC78 (ROS1+); KM12 (ALK+, TrkA+); BaF3/NTRK2:ETV6 (TrkB+); and BaF3/NTRK3:ETV6 (TrkC+). FIG. 2A: immunohistochemistry assay was performed with anti-ALK antibody D5F3®. The expected staining was observed in the spot corresponding to the sample derived from Karpas299 (ALK+, TrkA overexpression). FIG. 2B: immunohistochemistry assay was performed with anti-ROS1 antibody D4D5®. The expected staining was observed in the spot corresponding to the sample derived from HCC78 (ROS1+; dark spot, far right). The remaining samples were derived from the cell lines Karpas299, BaF3/NTRK2:ETV6, and KM12, and were negative. FIG. 2C: immunohistochemistry assay was performed with anti-Trk (pan) antibody C17F1®. The expected staining was observed in the spots corresponding to the samples derived from BaF3/NTRK2:ETV6, BaF3/NTRK3:ETV6, KM12, Karpas299, and ROS1.

FIGS. 3A and 3B depict the results of experiments demonstrating the performance of a multiplexed IHC assay performed on a tissue microarray (TMA) in accordance with some embodiments disclosed herein. A total of 72 samples derived from various tumor tissues were spotted on a microarray slide. Immunohistochemistry assay was performed with a mixture of three antibodies, D5F3®, D4D5®, C17F1®, as described in Example 2. As illustrated in FIG. 3A, positive staining observed in a number of tumor tissue samples which showed a range of staining intensities. FIG. 3B illustrates performance of a multiplexed IHC assay performed on adjacent tissue microarray (TMA) spots involving samples derived from various tumor populations.

FIG. 4A to 4E illustrate results of experiments demonstrating performance of multiplexed immunohistochemistry assays that were performed on various tissue samples FIG. 4A depicts differential expression of Trk in squamous lung carcinoma (right panel) and adenocarcinoma (left panel), as determined by an IHC assay using pan-Trk antibody C17F1®. FIG. 4B illustrates the correlation of positive staining and a known gene rearrangement, ETV6:NTRK3, which had been previously identified in secretory breast cancer tumor cells. Positive staining was observed with a mixture of three antibodies as described in Example 1 (middle panel) and anti-TrKC antibody (right panel), but negative staining was observed when the sample was individually stained for ALK and ROS1 (left panel). FIG. 4C illustrates the correlation of positive staining and a known gene arrangement, ETV6:NTRK3 gene fusion, which had been previously identified in papillary thyroid cancer cells. FIG. 4D illustrates the correlation of negative staining and lack of background staining in colorectal cancer cells. Tissues were stained with a mixture of three antibodies as described in Example 1 (top panel) and individual antibodies TrkA, TrkB, and TrkC (bottom panels). FIG. 4E illustrates the correlation of negative staining and lack of background staining in anaplastic large cell lymphoma (ALCL). Tissues were stained with a mixture of antibodies to ALK and ROS1 (top left panel), the mixture of three antibodies as described in Example 1 (top right panel), and individual antibodies to each of TrkA, TrkB and TrkC (bottom row).

### DETAILED DESCRIPTION

Disclosed is a multiplexed immunohistochemistry (IHC) assay that can be used in a wide range of research and clinical applications such as, for example, in methods for identifying and/or treating cancer patients harboring one or more molecular alterations in clinically important target biomarkers. In this assay, the one or more molecular alterations is detected by an antibody-based assay comprising one or more antibodies that bind to one or more of the target biomarkers. The multiplex IHC assay disclosed herein allows for rapid screening of large tissue populations to assess relative prevalence in multiple indications and can help guide future studies. In one embodiment, specimens selected as positive by the multiplexed IHC assay disclosed herein can be further tested in a second assay method with higher sensitivity and/or specificity such as, for example, fluorescent in-situ hybridization (FISH) or Next Generation Sequencing (NGS), depending on the variations being studied.

In the following detailed description, reference is made to the accompanying drawings, which form a part hereof. In the drawings, similar symbols typically identify similar components, unless context dictates otherwise. The illustrative alternatives described in the detailed description, drawings, and claims are not meant to be limiting. Other alternatives may be used, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are explicitly contemplated and make part of this disclosure.

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

### Some Definitions

As used herein, the term “N-[5-(3,5-difluorobenzyl)-1H-indazol-3-yl]-4-(piperazin-1-yl)-2-(tetrahydro-2H-pyran-4-ylamino) benzamide” means a compound having the chemical structure

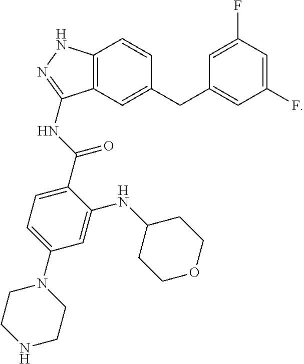 [SMILES: O=C(Nc1n[nH]c2ccc(Cc3cc(F)cc(F)c3)cc12)c1ccc(N2CCNCC2)cc1NC1CCOCC1]

As used herein, the term “N-[5-(3,5-difluorobenzyl)-1H-indazol-3-yl]-4-(4-methyl-piperazin-1-yl)-2-(tetrahydro-2H-pyran-4-ylamino) benzamide” means a compound having the chemical structure

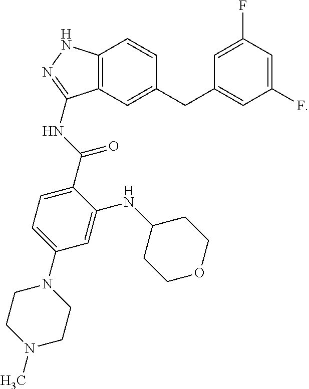 [SMILES: CN1CCN(c2ccc(C(=O)Nc3n[nH]c4ccc(Cc5cc(F)cc(F)c5)cc34)c(NC3CCOCC3)c2)CC1]

As used herein, the term “N-[5-(3,5-difluoro-benzenesulfonyl)-1H-indazol-3-yl]-2-((R)-2-methoxy-1-methyl-ethylamino)-4-(4-methyl-piperazin-1-yl) benzamide” means a compound having the chemical structure

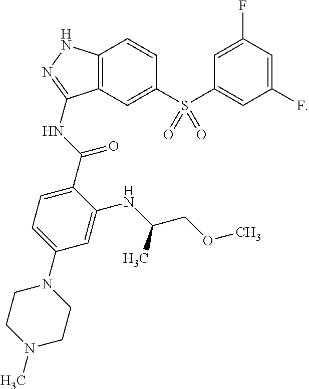 [SMILES: COC[C@@H](C)Nc1cc(N2CCN(C)CC2)ccc1C(=O)Nc1n[nH]c2ccc(S(=O)(=O)c3cc(F)cc(F)c3)cc12]

The singular form “a”, “an”, and “the” include plural references unless the context clearly dictates otherwise. For example, the term “a cell” includes one or more cells, including mixtures thereof “A and/or B” is used herein to include all of the following alternatives: “A”, “B”, “A or B”, and “A and B”.

“About” means either within plus or minus 10% of the provided value, or rounded to the nearest significant figure, in all cases inclusive of the provided value. Where ranges are provided, they are inclusive of the boundary values.

The terms “administration” and “administering” as used herein refer to the delivery of a bioactive composition or formulation by an administration route including, but not limited to, intravenous, intra-arterial, intramuscular, intraperitoneal, subcutaneous, intramuscular, topically, or combinations thereof.

As used herein, anaplastic lymphoma kinase (ALK) refers to ALK tyrosine kinase receptor or CD246 (cluster of differentiation 246), which is an enzyme that in humans is encoded by the ALK gene and also has the UniProt identified ALK_HUMAN.

As used herein, the term “antibody” refers to an immunoglobulin that specifically binds to, and is thereby defined as complementary with, a particular spatial and polar organization of another molecule. The antibody can be monoclonal or polyclonal and can be prepared by techniques that are well known in the art, such as immunization of a host and collection of sera (polyclonal), or by preparing continuous hybrid cell lines and collecting the secreted protein (monoclonal), or by cloning and expressing nucleotide sequences or mutagenized versions thereof coding at least for the amino acid sequences required for specific binding of natural antibodies. Antibodies may include a complete immunoglobulin or fragment thereof, which immunoglobulins include the various classes and isotypes, such as IgA, IgD, IgE, IgG1, IgG2a, IgG2b and IgG3, IgM, etc. Fragments thereof may include Fab, Fv and F(ab′)2, Fab′, and the like. In addition, aggregates, polymers, and conjugates of immunoglobulins or their fragments can be used where appropriate so long as binding affinity for a particular target is maintained.

The terms “monoclonal antibody,” “mAb” and “MAB” refer to an antibody that is an immunoglobulin produced by a single clone of lymphocytes which recognizes only a single epitope on an antigen. For example, a monoclonal antibody useful for the methods disclosed herein displays a single binding specificity and affinity for a particular epitope of one or more tyrosine kinases.

The term “polyclonal antibody” as used herein refers to a composition of different antibody molecules which is capable of binding to or reacting with several different specific antigenic determinants on the same or on different antigens. The variability in antigen specificity of a polyclonal antibody is located in the variable regions of the individual antibodies constituting the polyclonal antibody, in particular in the complementarity determining regions (CDRs). Preferably, the polyclonal antibody is prepared by immunization of an animal with the target tyrosine kinases or portions thereof. Alternatively, the polyclonal antibody may be prepared by mixing multiple monoclonal antibodies having desired specificity to a target tyrosine kinase.

The term “biological sample,” as used herein, encompasses a variety of sample types obtained from an organism and can be used in a diagnostic or monitoring assay. The sample may be of a healthy tissue, diseased tissue or tissue suspected of being diseased tissue. The sample may be a biopsy taken, for example, during a surgical procedure. The sample may be collected via means of fine needle aspiration, scraping or washing a cavity to collects cells or tissue therefrom. The sample may be of a tumor such as, for example, solid and hematopoietic tumors as well as of neighboring healthy tissue. The sample may be a smear of individual cells or a tissue section. The term encompasses blood and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain components. The term encompasses clinical samples, and also includes cells in cell culture, cell supernatants, cell lysates, cell extracts, cell homogenates, and subcellular components including synthesized proteins, serum, plasma, bodily and other biological fluids, and tissue samples. The biological sample can contain compounds that are not naturally intermixed with the cell or tissue in nature such as preservatives, anticoagulants, buffers, fixatives, nutrients, antibiotics or the like. In one embodiment, the sample is preserved as a frozen sample or as formaldehyde- or paraformaldehyde-fixed paraffin-embedded (FFPE) tissue preparation. For example, the sample can be embedded in a matrix, e.g., an FFPE block or a frozen sample.

The term “biomarker” is used herein to refer to a molecule whose level of nucleic acid or protein product has a quantitatively differential concentration or level with respect to an aspect of a biological state of a subject. “Biomarker” is used interchangeably with “marker” herein. The level of the biomarker can be measured at both the nucleic acid level as well as the polypeptide level. At the nucleic acid level, a nucleic acid gene or a transcript which is transcribed from any part of the subject's chromosomal and extrachromosomal genome, including for example the mitochondrial genome, may be measured. Preferably an RNA transcript, more preferably an RNA transcript includes a primary transcript, a spliced transcript, an alternatively spliced transcript, or an mRNA of the biomarker is measured. At the polypeptide level, a pre-propeptide, a propeptide, a mature peptide or a secreted peptide of the biomarker may be measured. A biomarker can be used either solely or in conjunction with one or more other identified biomarkers so as to allow correlation to the biological state of interest as defined herein. Specific examples of biomarkers covered by the present disclosure include ALK, ROS1, TrkA, TrkB, and TrkC.

The term “cancer” or “tumor” is used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers. In certain embodiments, the cancer is a solid tumor, a soft tissue tumor, or a metastatic lesion.

The term “chemotherapeutic agent”, as used herein, means a chemical substance, such as a cytotoxic or cytostatic agent, that is used to treat a condition, particularly cancer. In some embodiments, the chemotherapeutic agents include N-[5-(3,5-difluorobenzyl)-1H-indazol-3-yl]-4-(piperazin-1-yl)-2-(tetrahydro-2H-pyran-4-ylamino) benzamide, N-[5-(3,5-difluorobenzyl)-1H-indazol-3-yl]-4-(4-methyl-piperazin-1-yl)-2-(tetrahydro-2H-pyran-4-ylamino) benzamide, and N-[5-(3,5-difluoro-benzenesulfonyl)-1H-indazol-3-yl]-2-((R)-2-methoxy-1-methyl-ethylamino)-4-(4-methyl-piperazin-1-yl) benzamide, and pharmaceutically acceptable salts thereof.

As used herein the terms “combination” and “in combination with” mean the administration of a compound disclosed herein together with an at least one additional pharmaceutical or medicinal agent (e.g., an anti-cancer agent), either sequentially or simultaneously. It includes dosing simultaneously, or within minutes or hours of each other, or on the same day, or on alternating days, or dosing the compound disclosed herein on a daily basis, or multiple days per week, or weekly basis, for example, while administering another compound such as a chemotherapeutic agent on the same day or alternating days or weeks or on a periodic basis during a time simultaneous therewith or concurrent therewith, or at least a part of the time during which the compound disclosed herein is dosed. For example, the compound selected from N-[5-(3,5-difluorobenzyl)-1H-indazol-3-yl]-4-(piperazin-1-yl)-2-(tetrahydro-2H-pyran-4-ylamino) benzamide, N-[5-(3,5-difluorobenzyl)-1H-indazol-3-yl]-4-(4-methyl-piperazin-1-yl)-2-(tetrahydro-2H-pyran-4-ylamino) benzamide, and N-[5-(3,5-difluoro-benzenesulfonyl)-1H-indazol-3-yl]-2-((R)-2-methoxy-1-methyl-ethylamino)-4-(4-methyl-piperazin-1-yl) benzamide, or a pharmaceutically acceptable salt thereof could be dosed every day or several days a week while the chemotherapeutic agent is dosed on alternating days or alternating weeks or other periods of time, such as every 1, 2, 3, 4, 5, 6, 7, 8, 9, 10, 11, 12, 13, 14 or more days.

As used herein, “contact” in reference to specificity or specific binding means two molecules are close enough so that short range non-covalent chemical interactions, such as Van der Waal forces, hydrogen bonding, hydrophobic interactions, and the like, dominate the interaction of the molecule.

The term “cell line” as used herein refers to one or more generations of cells which are derived from a clonal cell. The term “clone,” or “clonal cell,” refers to a single cell which is expanded to produce an isolated population of phenotypically similar cells (i.e. a “clonal cell population”).

The term “immunohistochemistry”, as used herein, refers to the process of localizing antigens (e.g. proteins) in biological samples, cells and/or cells of a tissue section exploiting the principle of antibodies binding specifically to antigens. Immunohistochemical staining is widely used in the diagnosis of abnormal cells such as those found in cancerous tumors. Specific molecular markers are characteristic of particular cellular events, such as cell proliferation or cell death. Visualizing an antibody-antigen interaction can be accomplished in a number of ways. In the most common instance, an antibody is conjugated to an enzyme, such as peroxidase, that can catalyze a color-producing reaction. Alternatively, the antibody can also be tagged to a fluorophore thus employing the principles of immunofluorescence. Immunohistochemistry can also be used to evaluate tumor content in the sample on which qPCR is carried out in order to account for the fact that qPCR result will be influenced by the amount of tumor tissue present.

As used herein, the term “one or more molecular alterations” means any variation in the genetic or protein sequence in or more cells of a patient as compared to the corresponding wild-type genes or proteins. One or more molecular alterations include, but are not limited to, genetic mutations, gene amplifications, splice variants, deletions, insertions/deletions, gene rearrangements, single-nucleotide variations (SNVs), insertions, and aberrant RNA/protein expression.

A “multiplexed assay,” as used herein, refers to an assay in which multiple assay reactions, e.g. simultaneous assays of multiple target biomarkers, are carried out in a single reaction chamber and/or and analyzed in a single separation and detection format.

“Multiplex identification”, as used herein, refers to the simultaneous identification of one or more target biomarkers in a single mixture. For example, a two-plex assay refers to the simultaneous identification, in a single reaction mixture, of two different target biomarkers.

As used herein, “ROS1” refers to the ROS1 receptor tyrosine-protein kinase having the UniProt designation ROS1_HUMAN.

“Selectively binds” is defined as the situation in which one member of a specific intra- or inter-species binding pair will not show any significant binding to molecules other than its specific intra- or inter-species binding partner (e.g., an affinity of about 100-fold less), which means that only minimal cross-reactivity occurs.

“Specific”, as used herein in reference to the binding of two molecules or a molecule and a complex of molecules, refers to the specific recognition of one for the other and the formation of a stable complex, as compared to substantially less recognition of other molecules and the lack of formation of stable complexes with such other molecules. Preferably, “specific,” in reference to binding, means that to the extent that a molecule forms complexes with other molecules or complexes, it forms at least fifty percent of the complexes with the molecule or complex for which it has specificity. Generally, the molecules or complexes have areas on their surfaces or in cavities giving rise to specific recognition between the two binding moieties. Exemplary of specific binding are antibody-antigen interactions, enzyme-substrate interactions, polynucleotide hybridizations and/or formation of duplexes, cellular receptor-ligand interactions, and so forth.

As used herein, the term “therapeutically effective amount” means that amount of the compound or compounds being administered which will relieve to some extent one or more of the symptoms of the disorder being treated. In reference to the treatment of a cancer, a therapeutically effective amount refers to that amount which has the effect of (1) reducing the size of a cancer tumor, (2) inhibiting (that is, slowing to some extent, preferably stopping) cancer tumor metastasis, (3) inhibiting to some extent (that is, slowing to some extent, preferably stopping) cancer tumor growth, and/or, (4) relieving to some extent (or, preferably, eliminating) one or more symptoms associated with the cancer.

This amount will vary depending upon a variety of factors, including but not limited to the characteristics of the bioactive compositions and formulations disclosed herein (including activity, pharmacokinetics, pharmacodynamics, and bioavailability thereof), the physiological condition of the subject treated (including age, sex, disease type and stage, general physical condition, responsiveness to a given dosage, and type of medication) or cells, the nature of the pharmaceutically acceptable carrier or carriers in the formulation, and the route of administration. Further, an effective or therapeutically effective amount may vary depending on whether the one or more bioactive compositions and formulations disclosed herein is administered alone or in combination with other drug(s), other therapy/therapies or other therapeutic method(s) or modality/modalities. One skilled in the clinical and pharmacological arts will be able to determine an effective amount or therapeutically effective amount through routine experimentation, namely by monitoring a cell's or subject's response to administration of the one or more bioactive compositions and formulations disclosed herein and adjusting the dosage accordingly. A typical dosage may range from about 0.1 mg/kg to about 100 mg/kg or more, depending on the factors mentioned above. In other alternatives, the dosage may range from about 0.1 mg/kg to about 100 mg/kg; or about 1 mg/kg to about 100 mg/kg; or about 5 mg/kg up to about 100 mg/kg. For topical applications such as, for example, treatment of various hair conditions, according to some alternatives disclosed herein, suitable dosage may range from about 1 mg/kg to about 10 g/kg; or about 10 mg/kg to about 1 g/kg; or about 50 mg/kg up to about 10 g/kg. Additional guidance with regard to this aspect can be found in, for example, Remington: The Science and Practice of Pharmacy, 21st Edition, Univ. of Sciences in Philadelphia (USIP), Lippincott Williams & Wilkins, Philadelphia, Pa., 2005.

As used herein, the term “tropomyosin receptor kinase” means the family of tropomyosin receptor kinases (Trks) that are activated by peptide hormones of the neurotrophin family and include, but are not limited to, TrkA, TrkB, and TrkC. As used herein, the term “TrkA” means wild-type tropomyosin receptor kinase A having the UniProt identifier NTRK1_HUMAN. As used herein, the term “TrkB” means wild-type tropomyosin receptor kinase B having the UniProt identifier NTRK2_HUMAN. As used herein, the term “TrkC” means wild-type tropomyosin receptor kinase C having the UniProt identifier NTRK3_HUMAN. TrkA, TrkB and TrkC are also referred to by those having ordinary skill in the art as Trk1, Trk2 and Trk3, respectively. A reference to TrkA is a reference to Trk1. A reference to TrkB is a reference to Trk2. A reference to TrkC is a reference to Trk3.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non- limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Headings, e.g., (a), (b), (i) etc, are presented merely for ease of reading the specification and claims. The use of headings in the specification or claims does not require the steps or elements be performed in alphabetical or numerical order or the order in which they are presented.

Methods for selecting and Treating Cancer Patient

The preparation and use of compounds N-[5-(3,5-difluorobenzyl)-1H-indazol-3-yl]-4-(piperazin-1-yl)-2-(tetrahydro-2H-pyran-4-ylamino) benzamide and N-[5-(3,5-difluorobenzyl)-1H-indazol-3-yl]-4-(4-methyl-piperazin-1-yl)-2-(tetrahydro-2H-pyran-4-ylamino) benzamide as inhibitors of anaplastic lymphoma kinase are described in U.S. Pat. No. 8,299,057, issued Oct. 30, 2012, the disclosure of which is hereby incorporated by reference in its entirety.

The preparation and use of compound N-[5-(3,5-difluoro-benzenesulfonyl)-1H-indazol-3-yl]-2-((R)-2-methoxy-1-methyl-ethylamino)-4-(4-methyl-piperazin-1-yl) benzamide are described in U.S. Pat. No. 8,114,865, issued Feb. 14, 2012, the disclosure of which is hereby incorporated by reference in its entirety.

In one aspect, some embodiments disclosed herein relate to methods for treating cancer in a patient, including (a) acquiring knowledge of the presence of one or more molecular alterations in a biological sample from the cancer patient, wherein the one or more molecular alterations is detected by an assay comprising one or more antibodies that bind to one or more of ALK, ROS1, TrkA, TrkB, and TrkC biomarkers; (b) selecting a chemotherapeutic agent as a treatment for the cancer patient wherein the assay detects the presence of one or more of the one or more molecular alterations, and wherein the selected chemotherapeutic agent is one or more of N-[5-(3,5-difluorobenzyl)-1H-indazol-3-yl]-4-(piperazin-1-yl)-2-(tetrahydro-2H-pyran-4-ylamino) benzamide, N-[5-(3,5-difluorobenzyl)-1H-indazol-3-yl]-4-(4-methyl-piperazin-1-yl)-2-(tetrahydro-2H-pyran-4-ylamino) benzamide, and N-[5-(3,5-difluoro-benzenesulfonyl)-1H-indazol-3-yl]-2-((R)-2-methoxy-1-methyl-ethylamino)-4-(4-methyl-piperazin-1-yl) benzamide, or a pharmaceutically acceptable salt thereof and (c) administering a therapeutically effective amount of the one or more selected chemotherapeutic agents to the cancer patient. In some embodiments are provided such methods, wherein the selected chemotherapeutic agent is N-[5-(3,5-difluorobenzyl)-1H-indazol-3-yl]-4-(piperazin-1-yl)-2-(tetrahydro-2H-pyran-4-ylamino) benzamide, or a pharmaceutically acceptable salt thereof. In some embodiments are provided such methods, wherein the selected chemotherapeutic agent is N-[5-(3,5-difluorobenzyl)-1H-indazol-3-yl]-4-(4-methyl-piperazin-1-yl)-2-(tetrahydro-2H-pyran-4-ylamino) benzamide, or a pharmaceutically acceptable salt thereof. In some embodiments are provided such methods, wherein the selected chemotherapeutic agent is N-[5-(3,5-difluoro-benzenesulfonyl)-1H-indazol-3-yl]-2-((R)-2-methoxy-1-methyl-ethylamino)-4-(4-methyl-piperazin-1-yl) benzamide, or a pharmaceutically acceptable thereof.

Implementations of the methods according to this and other aspects of the present disclosure can include one or more of the following features. In some embodiments, the assay includes one or more antibodies that bind to at least two, three, four, or all of ALK, ROS1, TrkA, TrkB and TrkC biomarkers. In some embodiments, the one or more molecular alterations detected in the biological sample involve at least two, at least three, or at least four of the biomarkers. In some embodiments, the knowledge of the presence of the one or more molecular alterations in the biological sample is acquired from an assay that includes contacting the biological sample with one or more antibodies or fragments thereof that are specific for the biomarkers. In some embodiments, the specific antibodies are monoclonal antibodies. In some embodiments, the specific antibodies include at least one of D5F3®, D4D5®, C17F1®, and combinations thereof. In some embodiments, the biological sample is contacted with one or more of the specific antibodies simultaneously. In some embodiments, the biological sample is sequentially contacted with the specific antibodies. In some embodiments, the one or more molecular alterations results in elevated expression of one or more of the ALK, ROS1, TrkA, TrkB, and TrkC biomarkers. In some embodiments, the knowledge of the one or more molecular alterations is acquired from an assay wherein determining whether the expression of one or more biomarker is elevated includes: (a) determining the expression level of the one or more biomarkers in the biological sample; and (b) comparing the determined expression level to a reference expression level.

As used herein, the term “reference level” refers to known expression level of the target biomarker(s) in a control person or individual. In some embodiments, the reference expression level is the expression level of the target biomarker(s) in a healthy person or individual. In some embodiments, the reference expression level is the expression level of the target biomarker(s) in a population of healthy control cells. In some embodiments, the reference expression level is the expression level of the target biomarker(s) in a control person or individual that has been previously determined to possess one or more molecular alterations. In some embodiments, the reference expression level is the expression level of the target biomarker(s) in a population of control cells that have been previously determined to possess one or more molecular alterations.

In some embodiments, the knowledge of the one or more molecular alterations is acquired from an antibody-based assay. The antibody-based assay can generally be any antibody-based assay, and can be, for example, ELISA, immunohistochemistry, western blotting, mass spectrometry, flow cytometry, protein-microarray, immunofluorescence, and a multiplex detection assay. In some embodiments, the antibody-based assay includes an immunohistochemistry analysis.

In some embodiments, identifying a ALK, ROS1, TrkA, TrkB, or TrkC modulation defect such as an upregulation defect or a down-regulation defect, for example a null mutation such as a ALK, ROS1, TrkA, TrkB, or TrkC deletion or a ALK, ROS1, TrkA, TrkB, or TrkC chimeric locus encoding a constitutively active ALK, ROS1, TrkA, TrkB, or TrkC kinase in a cancer or precancerous pancreatic cell in an individual comprises assaying for ALK, ROS1, TrkA, TrkB, or TrkC activity in a cell extract from a pancreatic cancerous or precancerous cell population. In some embodiments, identifying a ALK, ROS1, TrkA, TrkB, or TrkC modulation defect such as an upregulation defect or a down-regulation defect, for example a null mutation such as a ALK, ROS1, TrkA, TrkB, or TrkC deletion or a ALK, ROS1, TrkA, TrkB, or TrkC chimeric locus encoding a constitutively active ALK, ROS1, TrkA, TrkB, or TrkC kinase in a cancer or precancerous pancreatic cell in an individual comprises assaying for ALK, ROS1, TrkA, TrkB, or TrkC transcript accumulation in an RNA population from a pancreatic cancerous or precancerous cell population. In some embodiments, identifying a ALK, ROS1, TrkA, TrkB, or TrkC modulation defect such as an upregulation defect or a down-regulation defect, for example a null mutation such as a ALK, ROS1, TrkA, TrkB, or TrkC deletion or a ALK, ROS1, TrkA, TrkB, or TrkC chimeric locus encoding a constitutively active ALK, ROS1, TrkA, TrkB, or TrkC kinase in a cancer or precancerous pancreatic cell in an individual comprises determining the nucleic acid sequence such as the genomic deoxyribonucleic acid sequence in a cell or cells or a cell population comprising a cell or cells from a pancreatic cancerous or precancerous cell population.

In some embodiments, methods of the present disclosure are to treat, reduce the symptoms of, ameliorate the symptoms of, delay the onset of, or otherwise pharmaceutically address a condition selected from non-small cell lung cancer, papillary thyroid cancer, neuroblastoma, pancreatic cancer and colorectal cancer and possibly other indications in which a defect in the modulation of ALK, ROS1, TrkA, TrkB, or TrkC activity, or a combination thereof, or upregulation, misregulation or deletion thereof might play a role by administering a molecule of U.S. Pat. No. 8,299,057, issued Oct. 30, 2012, the entirety of which is hereby incorporated by reference. In some embodiments, methods of the present disclosure are to treat, reduce the symptoms of, ameliorate the symptoms of, delay the onset of, or otherwise pharmaceutically address pancreatic cancer and possibly other indications in which a defect in the modulation of ROS1, TrkA, TrkB, or TrkC activity, or a combination thereof, activity, or upregulation, misregulation or deletion thereof might play a role by administering a molecule of U.S. Pat. No. 8,114,865, issued Feb. 14, 2012, the entirety of which is hereby incorporated by reference.

In some embodiments, methods of the present disclosure are to treat, reduce the symptoms of, ameliorate the symptoms of, delay the onset of, or otherwise pharmaceutically address a condition selected from non-small cell lung cancer, papillary thyroid cancer, neuroblastoma, pancreatic cancer and colorectal cancer associated with a ALK, ROS1, TrkA, TrkB, or TrkC down-regulation defect, for example a null mutation such as a ALK, ROS1, TrkA, TrkB, or TrkC deletion by identifying a ALK, ROS1, TrkA, TrkB, or TrkC down-regulation defect, for example a null mutation such as a ALK, ROS1, TrkA, TrkB, or TrkC deletion in a cancer or precancerous cell in an individual, and administering to the individual a compound as disclosed herein, such as N-[5-(3,5-difluorobenzyl)-1H-indazol-3-yl]-4-(piperazin-1-yl)-2-(tetrahydro-2H-pyran-4-ylamino) benzamide or N-[5-(3,5-difluorobenzyl)-1H-indazol-3-yl]-4-(4-methyl-piperazin-1-yl)-2-(tetrahydro-2H-pyran-4-ylamino) benzamide.

“Microarray,” as used herein, is an ordered arrangement of array elements (for example, small samples of a biological sample from a patient such as tissue samples) mounted on a solid support capable of binding other molecule species or antibodies. The array elements are arranged so that there are preferably at least one or more different array elements.

“Solid support,” as used herein, means the well-understood solid materials to which various components such as, for example, proteins and nucleic acids, are physically attached, thereby immobilizing the components. The term “solid support,” as used herein, means a non-liquid substance. A solid support can be, but is not limited to, a membrane, sheet, gel, glass, plastic or metal. Immobilized components may be associated with a solid support by covalent bonds and/or via non-covalent attractive forces such as hydrogen bond interactions, hydrophobic attractive forces and ionic forces, for example.

In some embodiments, the microarrays suitable for the methods disclosed herein have a density of at least 1, 2, 4, 6, 8, 10 spots/cm², preferably at least 10, 20, 30, 40, 50, 60, 70, 80, 90, 100, 110, 120, 130, 140, 150, 160, 170, 180, 190, 200, more preferably at least 210, 220, 230, 250, 275, 300, 325, 350, 375, 400, 425, 450, 475, 500, 550, 600, 650, 700, 750, 800, 850, 900, 950, 1000, 2000, 3000, 4000, 5000, 6000, 7000, 8000 or 9000 spots/cm².

In some embodiments, it is contemplated that the spots on the array may each represent a different species of biomarkers or that the multiple spots on the array may represent the same species of biomarkers. In some embodiments, the spots each represent an array element of differing identity or characteristics.

In some embodiments, implementations of the methods according to this and other aspects of the present disclosure further include acquiring knowledge of a genetic alteration in the cancer of the patient from a second analytical assay prior to the administering step. The second analytical assay can generally be any analytical assay known to those having ordinary skill in the art, and can be for example an antibody-based assay, a nucleotide-based assay, or an enzymatic activity assay. Non-limiting examples of suitable second analytical assays include capillary electrophoresis, nucleic acid sequencing, polypeptide sequencing, restriction digestion, nucleic acid amplification-based assays, nucleic acid hybridization assay, comparative genomic hybridization, real-time PCR, quantitative reverse transcription PCR (qRT-PCR), PCR-RFLP assay, HPLC, mass-spectrometric genotyping, fluorescent in-situ hybridization (FISH), next generation sequencing (NGS), and a kinase activity assay. Other examples of suitable second analytical assays include ELISA, immunohistochemistry, Western blotting, mass spectrometry, flow cytometry, protein-microarray, immunofluorescence, and multiplex detection assay.

In some embodiments, FISH analysis is used to identify the chromosomal rearrangement resulting in the one or more molecular alterations such as the fusion genes or gene products as described herein. For example, to perform FISH, at least a first probe tagged with a first detectable label can be designed to target a first gene of a fusion gene, such as in one or more exons of the gene and at least a second probe tagged with a second detectable label can be designed to target a second gene of the fusion gene, such as in one or more exons of the genes (for example, the exons containing the part of the protein that includes the tyrosine kinase domain). The at least one first probe and the at least one second probe will be closer together in a subject who carries the fusion compared to a subject who does not carry the fusion gene or gene product. In some embodiments, a variation of a FISH assay, for example, “break-apart FISH”, is used to evaluate a patient selected by a method disclosed herein. By this method, at least one probe targeting the fusion junction and at least one probe targeting an individual gene of the fusion, e.g., at one or more exons and or introns of the gene, are utilized. In normal cells, both probes will be observed (or a secondary color will be observed due to the close proximity of the two genes of the gene fusion), and only the single gene probe will be observed when the translocation occurs or the probes, having differing colors, will be separated such that one of ordinary skill in the art observing the probes can determine that a relevant gene fusion or deletion is present in the sample. Generally, FISH assays are performed using formalin-fixed, paraffin-embedded tissue sections that are placed on slides. The DNA from the tissue sample sections is denatured to single-stranded form and subsequently allowed to hybridize with the appropriate DNA probes that can be designed and prepared using methods and techniques known to those having ordinary skill in the art. Following hybridization, any unbound probe may be removed by a series of washes and the nuclei of the cells are counter-stained with DAPI (4′,6 diamidino-2-phenylindole), a DNA-specific stain that fluoresces blue. Hybridization of the probe or probes are viewed using a fluorescence microscope equipped with appropriate excitation and emission filters, allowing visualization of the fluorescent signals.

For example, a break-apart FISH assay may be used to detect multiple types of rearrangements involving the ALK gene locus. In the method, tumor cells from some patients having non-small cell lung cancer (NSCLC), display an ALK-positive FISH pattern as detected using single interference filter sets comprising green (FITC), red (Texas red), and blue (4′,6-diamidino-2-phenylindole) as well as dual (red/green) and triple (blue, red, green) band-pass filters. A fusion of the ALK gene is visualized as split orange and green signals, single orange signals, or single orange and single green signals.

Relevant molecular alterations with respect to ROS1, TrkA, TrkB and TrkC in biological samples derived from cancer patients using the same methods as described above, but by modifying the reagents, probes and other materials used in the assays in ways that are appropriate to the target molecular alteration and as can be readily determined by those having ordinary skill in the art.

Other variations of the FISH method known in the art are suitable for evaluating a patient selected in accordance with the methods disclosed herein.

In some embodiments of the methods disclosed herein, the cancer is selected from the group consisting of anaplastic large-cell lymphoma (ALCL), colorectal cancer (CRC), cholangiocarcinoma, gastric, glioblastomas (GBM), leiomyosarcoma, melanoma, non-small cell lung cancer (NSCLC), squamous cell lung cancer, neuroblastoma (NB), ovarian cancer, pancreatic cancer, prostate cancer, medullary thyroid cancer, breast cancer, and papillary thyroid cancer. In some embodiments are provided such methods, wherein the knowledge of the presence of the one or more molecular alterations is obtained from an assay performed simultaneously on a plurality of biological samples. In some embodiments, the plurality of biological samples may be assayed in a multitest platform.

As used herein, the term “multitest platform” is intended to encompass any suitable means to contain one or more reaction mixtures, suspensions, or detection reactions. As such, the outcomes of a number of screening events can be assembled onto one surface, resulting in a “multitest platform” having, or consisting of multiple elements or parts to do more than one experiment simultaneously. It is intended that the term “multitest platform” encompasses protein chips, microtiter plates, multi-well plates, microcards, test tubes, petri plates, trays, slides, and the like. In some embodiments, multiplexing can further include simultaneously conducting a plurality of screening events in each of a plurality of separate biological samples. For example, the number of biological samples analyzed can be based on the number of spots on a slide and the number of tests conducted in each spot (as described in greater detail in Example 2). In another example, the number of biological samples analyzed can be based on the number of wells in a multi-well plate and the number of tests conducted in each well. For example, 6-well, 12-well, 24-well, 48-well, 96-well, 384-well, 1536-well or 3456-well microtiter plates can be useful in the presently disclosed methods, although it will be appreciated by those in the art, not each microtiter well need contain an individual biological sample. Depending on the size of the microtiter plate and the number of the individual biological samples in each well, very high numbers of tests can be run simultaneously. Although multiplexing has been exemplified in Example 2 with respect to micro-slides, it will be understood that other formats can be used for multiplexing.

In some embodiments are provided such methods, wherein the plurality of biological samples includes at least 6, 12, 24, 48, 96, 200, 384, 400, 500, 1000, 1250, 1500, or 3000 samples.

In some embodiments are provided such methods, wherein the one or more molecular alterations is selected from a genetic mutation, a gene amplification, a gene rearrangement, a single-nucleotide variation (SNV), a deletion, an insertion, an InDel mutation, a single nucleotide point mutation (SNP), an epigenetic alteration, a splicing variant, an RNA/protein overexpression, and an aberrant RNA/protein expression. In some embodiments are provided such methods, wherein the genetic alteration includes an insertion of a heterologous nucleic acid sequence within a coding sequence of a biomarker gene. In some embodiments are provided such methods, wherein the insertion forms a chimeric nucleic acid sequence that encodes a fusion peptide.

In some embodiments are provided such methods, wherein the acquiring knowledge of the one or more molecular alterations further comprises determining a nucleic acid sequence and/or an amino acid sequence comprising the one or more molecular alterations. In some embodiments, the nucleic acid sequence comprising the one or more molecular alterations from a selected cancer patient tumor is sequenced. In some embodiments, the sequence is determined by a next generation sequencing method.

Some embodiments of the methods disclosed herein comprise selecting one or more chemotherapeutic agents appropriate for the treatment of the cancer, and administering a therapeutically effective amount of the selected one or more chemotherapeutic agents to the patient. Non-limiting examples of such chemotherapeutic agents include those listed in TABLE 1, or any pharmaceutically acceptable salt thereof. In some embodiments, the selected chemotherapeutic agent is selected from the group consisting of N-[5-(3,5-difluorobenzyl)-1H-indazol-3-yl]-4-(piperazin-1-yl)-2-(tetrahydro-2H-pyran-4-ylamino) benzamide, N-[5-(3,5-difluorobenzyl)-1 H-indazol-3-yl]-4-(4-methyl-piperazin-1-yl)-2-(tetrahydro-2H-pyran-4-ylamino) benzamide, and N-[5-(3,5-difluoro-benzenesulfonyl)-1H-indazol-3-yl]-2-((R)-2-methoxy-1-methyl-ethylamino)-4-(4-methyl-piperazin-1-yl) benzamide, and any pharmaceutically acceptable salt thereof.

TABLE 1
Non-limiting examples of chemotherapeutic agents

| Compound Name | CAS Registry No. | Chemical Name | Reference |
|---|---|---|---|
| crizotinib | 877399-52-5 | (R)-3-[1-(2,6-Dichloro-3- | U.S. |
| | | fluorophenyl)ethoxy]-5-[1-(piperidin-4- | Pat. No. |
| | | yl)-1H-pyrazol-4-yl]pyridin-2-amine | 7,230,098 |
| entrectinib | 1108743-60-7 | N-[5-(3,5-difluorobenzyl)-1H-indazol-3- | U.S. |
| | | yl]-4-(4-methyl-piperazin-1-yl)-2- | Pat. No. |
| | | (tetrahydro-pyran-4-ylamino)-benzamide | 8,299,057 |
| NVP-TAE684 | 761439-42-3 | 5-chloro-N2-[2-methoxy-4-[4-(4-methyl- | U.S. |
| | | 1-piperazinyl)-1-piperidinyl]phenyl]-N4- | Pat. No. |
| | | [2-[(1-methylethyl)sulfonyl]phenyl]-2,4- | 7,964,592 |
| | | Pyrimidinediamine | |
| foretinib | 937176-80-2 | 1-N′-[3-fluoro-4-[6-methoxy-7-(3- | U.S. |
| | | morpholin-4-ylpropoxy)quinolin-4- | Pat. No. |
| | | yl]oxyphenyl]-1-N-(4- | 8,497,284 |
| | | fluorophenyl)cyclopropane-1,1- | |
| | | dicarboxamide | |
| BMS-754807 | 1001350-96-4 | (2S)-1-[4-[(5-cyclopropyl-1H-pyrazol-3- | U.S. |
| | | yl)amino]pyrrolo[2,1-f][1,2,4]triazin-2- | Pat. No. |
| | | yl]-N-(6-fluoropyridin-3-yl)-2- | 7,534,792 |
| | | methylpyrrolidine-2-carboxamide | |
| GNF 5837 | 1033769-28-6 | 1-[2-fluoro-5-(trifluoromethyl)phenyl]-3- | WO 2008073480 |
| | | [4-methyl-3-[[(3Z)-2-oxo-3-(1H-pyrrol-2- | |
| | | ylmethylidene)-1H-indol-6- | |
| | | yl]amino]phenyl]urea | |
| rebastinib | 1020172-07-9 | 4-[4-[(5-tert-butyl-2-quinolin-6-ylpyrazol- | U.S. |
| | | 3-yl)carbamoylamino]-3-fluorophenoxy]- | Pat. No. |
| | | N-methylpyridine-2-carboxamide | 7,790,756 |
| GW441756 | 504433-23-2 | 3-[(1-methylindol-3-yl)methylidene]-1H- | U.S. |
| | | pyrrolo[3,2-b]pyridin-2-one | Pat. No. |
| | | | 7,015,231 |
| cabozantinib | 849217-68-1 | 1-N-[4-(6,7-dimethoxyquinolin-4- | U.S. |
| | | yl)oxyphenyl]-1-N′-(4- | Pat. No. |
| | | fluorophenyl)cyclopropane-1,1- | 7,579,473 |
| | | dicarboxamide | |
| bosutinib | 380843-75-4 | 4-(2,4-dichloro-5-methoxyanilino)-6- | WO 2004075898 |
| | | methoxy-7-[3-(4-methylpiperazin-1- | |
| | | yl)propoxy]quinoline-3-carbonitrile | |
| Compound 2 | 1034974-86-1 | N-[5-(3,5-difluoro-benzenesulfonyl)-1H- | U.S. |
| | | indazol-3-yl]-2-((R)-2-methoxy-1-methyl- | Pat. No. |
| | | ethylamino)-4-(4-methyl-piperazin-1-yl)benzamide | 8,114,865 |
| TSR-011 | 1388225-79-3 | N-[1,3-dihydro-6-[[4-(1-hydroxy-1- | Journal of |
| | | methylethyl)-1-piperidinyl]methyl]-1-[cis- | Medicinal |
| | | 4-[[(1- | Chemistry, |
| | | methylethyl)amino]carbonyl]cyclohexyl]- | Volume 55, |
| | | 2H-benzimidazol-2-ylidene]-3,5-difluoro-, | Issue 14, |
| | | [N(E)]-benzamide, | pp. 6523-6540, |
| | | | 2012 |
| MGCD516 | 1123837-84-2 | N-[3-fluoro-4-[[2-[5-[[(2- | U.S. |
| | | methoxyethyl)amino]methyl]-2- | Pat. No. |
| | | pyridinyl]thieno[3,2-b]pyridin-7- | 8,404,846 |
| | | yl]oxy]phenyl]-N′-(4-fluorophenyl)-1,1- | |
| | | cyclopropanedicarboxamide | |
| ceritinib | 1032900-25-6 | 5-chloro-2-N-(5-methyl-4-piperidin-4-yl- | U.S. |
| | | 2-propan-2-yloxyphenyl)-4-N-(2-propan- | Pat. No. |
| | | 2-ylsulfonylphenyl)pyrimidine-2,4- | 8,372,858 |
| | | diamine | |
| LOXO-101 | 1223403-58-4 | (3S) - N-[5-[(2R)-2-(2,5-difluorophenyl)-1- | U.S. |
| | | pyrrolidinyl]pyrazolo[1,5-a]pyrimidin-3- | Pat. No. |
| | | yl]-3-hydroxy-1-pyrrolidinecarboxamide | 8,513,263 |
| PF-06463922 | 1454846-35-5 | (10R)-7-amino-12-fluoro-10,15,16,17- | U.S. |
| | | tetrahydro-2,10,16-trimethyl-15-oxo-2H- | Pat. No. |
| | | 4,8-Methenopyrazolo[4,3- | 8,680,111 |
| | | h][2,5,11]benzoxadiazacyclotetradecine- | |
| | | 3-carbonitrile | |
| AZ-23 | 915720-21-7 | 5-chloro-2-N-[(1S)-1-(5-fluoropyridin-2- | U.S. |
| | | yl)ethyl]-4-N-(3-propan-2-yloxy-1H- | Pat. No. |
| | | pyrazol-5-yl)pyrimidine-2,4-diamine | 8,1149,89 |
| K252a | 99533-80-9 | 9,12-Epoxy-1H-diindolo[1,2,3-fg:3′,2′,1′- | U.S. |
| | | kl]pyrrolo[3,4-i][1,6]benzodiazocine-10- | Pat. No. |
| | | carboxylic acid, 2,3,9,10,11,12- | 4,555,402 |
| | | hexahydro-10-hydroxy-9-methyl-1-oxo-, | |
| | | methyl ester, (9S,10R,12R)- | |
| Staurosporine | 62996-74-1 | 9,13-Epoxy-1H,9H-diindolo[1,2,3- | Commercially |
| | | gh:3′,2′,1′-lm]pyrrolo[3,4- | available; |
| | | j][1,7]benzodiazonin-1-one, | Journal of |
| | | 2,3,10,11,12,13-hexahydro-10-methoxy- | Antibiotics |
| | | 9-methyl-11-(methylamino)-, | Volume 30, |
| | | (9S,10R,11R,13R)- | Issue 4, |
| | | | pp.75-82, 1977 |

In some embodiments of the methods disclosed herein, at least one of the chemotherapeutic agents N-[5-(3,5-difluorobenzyl)-1H-indazol-3-yl]-4-(piperazin-1-yl)-2-(tetrahydro-2H-pyran-4-ylamino) benzamide, N-[5-(3,5-difluorobenzyl)-1H-indazol-3-yl]-4-(4-methyl-piperazin-1-yl)-2-(tetrahydro-2H-pyran-4-ylamino) benzamide, and N-[5-(3,5-difluoro-benzenesulfonyl)-1H-indazol-3-yl]-2-((R)-2-methoxy-1-methyl-ethylamino)-4-(4-methyl-piperazin-1-yl) benzamide, or a pharmaceutically acceptable salt thereof, are selected for administration or are administered to an individual or patient having cancer, optionally in combination with at least one additional chemotherapeutic agent.

In some embodiments of the methods disclosed herein, at least one of the chemotherapeutic agents N-[5-(3,5-difluorobenzyl)-1H-indazol-3-yl]-4-(piperazin-1-yl)-2-(tetrahydro-2H-pyran-4-ylamino) benzamide, N-[5-(3,5-difluorobenzyl)-1H-indazol-3-yl]-4-(4-methyl-piperazin-1-yl)-2-(tetrahydro-2H-pyran-4-ylamino) benzamide, and N-[5-(3,5-difluoro-benzenesulfonyl)-1H-indazol-3-yl]-2-((R)-2-methoxy-1-methyl-ethylamino)-4-(4-methyl-piperazin-1-yl) benzamide, or a pharmaceutically acceptable salt thereof, are administered to a patient or individual having or suffering from cancer in an amount ranging from about 200 mg/m2 to about 1600 mg/m2, or from about 200 mg/m2 to about 1200 mg/m2, or from about 200 mg/m2 to about 1000 mg/m2, or from about 400 mg/m2 to about 1200 mg/m2, or from about 400 mg/m2 to about 1000 mg/m2, or from about 800 mg/m2 to about 1000 mg/m2, or from about 800 mg/m2 to about 1200 mg/m2, or from about 800 mg/m2 to about 1200 mg/m2, or from about 800 mg/m2 to about 1600 mg/m2. In some embodiments, at least one of the chemotherapeutic agents described above are administered to the individual in an amount of about 200 mg/m2, about 300 mg/m2, about 400 mg/m2, about 500 mg/m2, about 600 mg/m2, about 700 mg/m2, about 800 mg/m2, about 900 mg/m2, about 1000 mg/m2, about 1100 mg/m2, about 1200 mg/m2, about 1300 mg/m2, about 1400 mg/m2, about 1500 mg/m2, about 1600 mg/m2, about 1700 mg/m2, about 1800 mg/m2, about 1900 mg/m2, or about 2000 mg/m2. In some embodiments, the selected chemotherapeutic agent, or a pharmaceutically accepted salt thereof, is administered to a patient or individual having or suffering from cancer in multiple dosages for a treatment period of 2 to 50 days. In some embodiments, the selected chemotherapeutic agent, or a pharmaceutically accepted salt thereof, is administered to a patient or individual having or suffering from cancer in multiple dosages of about 50 to about 200 mg/kg per dose over a treatment period of 5 to 42 days. In some embodiments, the selected chemotherapeutic agent, or a pharmaceutically accepted salt thereof, is administered to a patient or individual having or suffering from cancer with an oral dosage of about 60 mg/kg twice a day (BID), seven times per week. In some embodiments, the selected chemotherapeutic agent, or a pharmaceutically accepted salt thereof, is administered to a patient or individual having or suffering from cancer with an oral dosage of about 60 mg/kg twice a day (BID), seven times per week for six weeks, on alternate weekly basis (i.e. one week on one week off).

Some embodiments include any of the methods described herein, wherein at least one of the compounds N-[5-(3,5-difluorobenzyl)-1H-indazol-3-yl]-4-(piperazin-1-yl)-2-(tetrahydro-2H-pyran-4-ylamino) benzamide, N-[5-(3,5-difluorobenzyl)-1H-indazol-3-yl]-4-(4-methyl-piperazin-1-yl)-2-(tetrahydro-2H-pyran-4-ylamino) benzamide, and N-[5-(3,5-difluoro-benzenesulfonyl)-1H-indazol-3-yl]-2-((R)-2-methoxy-1-methyl-ethylamino)-4-(4-methyl-piperazin-1-yl) benzamide, or a pharmaceutically acceptable salt thereof, are administered to a patient or individual having or suffering from cancer in an amount ranging from about 0.01 mg /kg to about 100 mg/kg, or from about 0.02 mg/kg to about 50 mg/kg, or from about 0.05 mg/kg to about 25 mg/kg, or from about 0.1 mg/kg to about 20 mg/kg, or from about 0.2 mg/kg to about 10 mg/kg, or from about 0.5 mg/kg to about 5 mg/kg, or from about 1 mg/kg to about 2 mg/kg.

The therapeutic agents disclosed herein may be administered to a cancer patient in need thereof by administration to the patient of a pharmaceutical composition comprising one or more such agents. In particular, such pharmaceutical compositions may comprise one or more of a compound selected from N-[5-(3,5-difluorobenzyl)-1H-indazol-3-yl]-4-(piperazin-1-yl)-2-(tetrahydro-2H-pyran-4-ylamino) benzamide, N-[5-(3,5-difluorobenzyl)-1H-indazol-3-yl]-4-(4-methyl-piperazin-1-yl)-2-(tetrahydro-2H-pyran-4-ylamino) benzamide, and N-[5-(3,5-difluoro-benzenesulfonyl)-1H-indazol-3-yl]-2-((R)-2-methoxy-1-methyl-ethylamino)-4-(4-methyl-piperazin-1-yl) benzamide, or a pharmaceutically acceptable salt thereof, and at least one pharmaceutically acceptable excipient. In some embodiments are provided such pharmaceutical compositions comprising N-[5-(3,5-difluorobenzyl)-1H-indazol-3-yl]-4-(piperazin-1-yl)-2-(tetrahydro-2H-pyran-4-ylamino) benzamide, or a pharmaceutically acceptable salt thereof, and at least one pharmaceutically acceptable excipient. In some embodiments are provided such pharmaceutical compositions wherein the compound is N-[5-(3,5-difluorobenzyl)-1H-indazol-3-yl]-4-(4-methyl-piperazin-1-yl)-2-(tetrahydro-2H-pyran-4-ylamino) benzamide, or a pharmaceutically acceptable salt thereof, and at least one pharmaceutically acceptable excipient. In some embodiments are provided such pharmaceutical compositions wherein the compound is N-[5-(3,5-difluoro-benzenesulfonyl)-1H-indazol-3-yl]-2-((R)-2-methoxy-1-methyl-ethylamino)-4-(4-methyl-piperazin-1-yl) benzamide, or a pharmaceutically acceptable salt thereof, and at least one pharmaceutically acceptable excipient.

Some embodiments of the pharmaceutical compositions can comprise a physical admixture of the various ingredients in solid, liquid, or gelcap form. Other embodiments can comprise at least two separated ingredients in a single dosage unit or dosage form, such as, for example, a two- or three-layer tablet in which at least two active ingredients are located in separate layers or regions of the tablet, optionally separated by a third material, such as, for example, a sugar layer or other inert barrier to prevent contact between the first two ingredients. In other embodiments, two or more active ingredients are separately formulated into individual dosage units, which are then packaged together for ease of administration. One embodiment comprises a package containing a plurality of individual dosage units. This embodiment may, for example, comprise a blister package. In one embodiment of a blister package, multiple blister-packed dosage units are present on a single sheet, and those units that are to be administered together are packaged in the same or adjacent blisters of the blister pack. Alternatively, any other packaging can be used in which two active ingredients are packaged together for concurrent or sequential use.

Some embodiments relate to the use of any of the compounds as described herein, or a pharmaceutically acceptable salt thereof, in the manufacture of a medicament for the treatment of abnormal cell growth in a mammal. The present disclosure further relates to the use of any of the compounds as described herein, or a pharmaceutically acceptable salt thereof, in the manufacture of a medicament for the treatment of abnormal cell growth in a mammal wherein the abnormal cell growth is cancerous or non-cancerous. In some embodiments, the abnormal cell growth is cancerous. In another embodiment, the abnormal cell growth is non-cancerous.

Some embodiments relate to any of the compounds described herein, or pharmaceutically acceptable salts thereof, for use as a medicament. Some embodiments relate to the use of any of the compounds described above, or pharmaceutically acceptable salts thereof, for the manufacture of a medicament for the treatment of abnormal cell growth.

As used herein “cancer” refers to any malignant and/or invasive growth or tumor caused by abnormal cell growth. As used herein “cancer” refers to solid tumors named for the type of cells that form them, cancer of blood, bone marrow, or the lymphatic system. Examples of solid tumors include but are not limited to sarcomas and carcinomas. Examples of cancers of the blood include but are not limited to leukemias, lymphomas and myeloma.

The term “cancer” includes but is not limited to a primary cancer that originates at a specific site in the body, a metastatic cancer that has spread from the place in which it started to other parts of the body, a recurrence from the original primary cancer after remission, and a second primary cancer that is a new primary cancer in a person with a history of previous cancer of different type from latter one.

Some embodiments relate to compositions comprising a compound selected from N-[5-(3,5-difluorobenzyl)-1H-indazol-3-yl]-4-(piperazin-1-yl)-2-(tetrahydro-2H-pyran-4-ylamino) benzamide, N-[5-(3,5-difluorobenzyl)-1H-indazol-3-yl]-4-(4-methyl-piperazin-1-yl)-2-(tetrahydro-2H-pyran-4-ylamino) benzamide, and N-[5-(3,5-difluoro-benzenesulfonyl)-1H-indazol-3-yl]-2-((R)-2-methoxy-1-methyl-ethylamino)-4-(4-methyl-piperazin-1-yl) benzamide, or a pharmaceutically acceptable salt thereof (e.g., pharmaceutical compositions). Accordingly, in some embodiments, the present disclosure relates to a pharmaceutical composition comprising a compound selected from N-[5-(3,5-difluorobenzyl)-1H-indazol-3-yl]-4-(piperazin-1-yl)-2-(tetrahydro-2H-pyran-4-ylamino) benzamide, N-[5-(3,5-difluorobenzyl)-1H-indazol-3-yl]-4-(4-methyl-piperazin-1-yl)-2-(tetrahydro-2H-pyran-4-ylamino) benzamide, and N-[5-(3,5-difluoro-benzenesulfonyl)-1H-indazol-3-yl]-2-((R)-2-methoxy-1-methyl-ethylamino)-4-(4-methyl-piperazin-1-yl) benzamide, or a pharmaceutically acceptable salt, a pharmaceutically acceptable carrier and, optionally, at least one additional medicinal or pharmaceutical agent. In some embodiments, the at least one additional medicinal or pharmaceutical agent is an anti-cancer agent as described below. In some embodiments, the compound is N-[5-(3,5-difluorobenzyl)-1H- indazol-3-yl]-4-(piperazin-1-yl)-2-(tetrahydro-2H-pyran-4-ylamino) benzamide, or a pharmaceutically acceptable salt thereof. In some embodiments, the compound is N-[5-(3,5-difluorobenzyl)-1H-indazol-3-yl]-4-(4-methyl-piperazin-1-yl)-2-(tetrahydro-2H-pyran-4-ylamino) benzamide, or a pharmaceutically acceptable salt thereof. In some embodiments, the compound is N-[5-(3,5-difluoro-benzenesulfonyl)-1H-indazol-3-yl]-2-((R)-2-methoxy-1-methyl-ethylamino)-4-(4-methyl-piperazin-1-yl) benzamide, or a pharmaceutically acceptable salt thereof. In some embodiments, the compound is at least two of N-[5-(3,5-difluorobenzyl)-1H-indazol-3-yl]-4-(piperazin-1-yl)-2-(tetrahydro-2H-pyran-4-ylamino) benzamide, N-[5-(3,5-difluorobenzyl)-1H-indazol-3-yl]-4-(4-methyl-piperazin-1-yl)-2-(tetrahydro-2H-pyran-4-ylamino) benzamide, and N-[5-(3,5-difluoro-benzenesulfonyl)-1H-indazol-3-yl]-2-((R)-2-methoxy-1-methyl-ethylamino)-4-(4-methyl-piperazin-1-yl) benzamide, or a pharmaceutically acceptable salt thereof.

The pharmaceutically acceptable carrier may comprise a conventional pharmaceutical carrier or excipient. Suitable pharmaceutical carriers include inert diluents or fillers, water and various organic solvents (such as hydrates and solvates). The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Non-limiting examples of materials, therefore, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof

The pharmaceutical composition may, for example, be in a form suitable for oral administration as a tablet, capsule, pill, powder, sustained release formulations, solution suspension, for parenteral injection as a sterile solution, suspension or emulsion, for topical administration as an ointment or cream or for rectal administration as a suppository.

Exemplary parenteral administration forms include solutions or suspensions of active compounds in sterile aqueous solutions, for example, aqueous propylene glycol or dextrose solutions. Such dosage forms may be suitably buffered, if desired.

The pharmaceutical composition may be in unit dosage forms suitable for single administration of precise dosages.

In some embodiments, the composition comprises a therapeutically effective amount of a compound as disclosed herein and a pharmaceutically acceptable carrier.

The compounds of the present disclosure may be formulated into pharmaceutical compositions as described below in any pharmaceutical form recognizable to the skilled artisan as being suitable. Pharmaceutical compositions of the disclosure comprise a therapeutically effective amount of at least one compound disclosed herein and an inert, pharmaceutically acceptable carrier or diluent.

To treat or prevent diseases or conditions mediated by ALK, ROS1, TrkA, TrkB, or TrkC, or a combination thereof, a pharmaceutical composition of the present disclosure is administered in a suitable formulation prepared by combining a therapeutically effective amount (i.e. , a ALK, ROS1, TrkA, TrkB, or TrkC modulating, regulating, or inhibiting amount effective to achieve therapeutic efficacy) of at least one compound of the present disclosure (as an active ingredient) with one or more pharmaceutically suitable carriers, which may be selected, for example, from diluents, excipients and auxiliaries that facilitate processing of the active compounds into the final pharmaceutical preparations.

The pharmaceutical carriers employed may be either solid or liquid. Exemplary solid carriers are lactose, sucrose, talc, gelatin, agar, pectin, acacia, magnesium stearate, stearic acid and the like. Exemplary liquid carriers are syrup, peanut oil, olive oil, water and the like. Similarly, the inventive compositions may include time-delay or time- release material known in the art, such as glyceryl monostearate or glyceryl distearate alone or with a wax, ethylcellulose, hydroxypropylmethylcellulose, methylmethacrylate or the like. Further additives or excipients may be added to achieve the desired formulation properties. For example, a bioavailability enhancer, such as Labrasol, Gelucire or the like, or formulator, such as CMC (carboxy-methylcellulose), PG (propyleneglycol), or PEG (polyethyleneglycol), may be added. Gelucire®, a semi-solid vehicle that protects active ingredients from light, moisture and oxidation, may be added, e.g., when preparing a capsule formulation.

If a solid carrier is used, the preparation can be tableted, placed in a hard gelatin capsule in powder or pellet form, or formed into a troche or lozenge. The amount of solid carrier may vary, but generally will be from about 25 mg to about 1 g. If a liquid carrier is used, the preparation may be in the form of syrup, emulsion, soft gelatin capsule, sterile injectable solution or suspension in an ampoule or vial or non-aqueous liquid suspension. If a semi-solid carrier is used, the preparation may be in the form of hard and soft gelatin capsule formulations. The inventive compositions are prepared in unit-dosage form appropriate for the mode of administration, e.g. parenteral or oral administration.

To obtain a stable water-soluble dose form, a salt of a compound of the present disclosure may be dissolved in an aqueous solution of an organic or inorganic acid, such as a 0.3 M solution of succinic acid or citric acid. If a soluble salt form is not available, the agent may be dissolved in a suitable co-solvent or combinations of co-solvents. Examples of suitable co-solvents include alcohol, propylene glycol, polyethylene glycol 300, polysorbate 80, glycerin and the like in concentrations ranging from 0 to 60% of the total volume. In an exemplary embodiment, a compound of the present disclosure is dissolved in DMSO and diluted with water. The composition may also be in the form of a solution of a salt form of the active ingredient in an appropriate aqueous vehicle such as water or isotonic saline or dextrose solution.

Proper formulation is dependent upon the route of administration selected. For injection, the agents of the compounds of the present disclosure may be formulated into aqueous solutions, preferably in physiologically compatible buffers such as Hanks solution, Ringer's solution, or physiological saline buffer. For transmucosal administration, penetrants appropriate to the barrier to be permeated are used in the formulation. Such penetrants are generally known in the art.

For oral administration, the compounds can be formulated by combining the active compounds with pharmaceutically acceptable carriers known in the art. Such carriers enable the compounds of the disclosure to be formulated as tablets, pills, dragees, capsules, liquids, gels, syrups, slurries, suspensions and the like, for oral ingestion by a subject to be treated. Pharmaceutical preparations for oral use can be obtained using a solid excipient in admixture with the active ingredient (agent), optionally grinding the resulting mixture, and processing the mixture of granules after adding suitable auxiliaries, if desired, to obtain tablets or dragee cores. Suitable excipients include: fillers such as sugars, including lactose, sucrose, mannitol, or sorbitol; and cellulose preparations, for example, maize starch, wheat starch, rice starch, potato starch, gelatin, gum, methyl cellulose, hydroxypropylmethyl- cellulose, sodium carboxymethylcellulose, or polyvinylpyrrolidone (PVP). If desired, disintegrating agents may be added, such as crosslinked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active agents.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate, and, optionally, stabilizers. In soft capsules, the active agents may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

For administration intranasally or by inhalation, the compounds for use according to the present disclosure may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of gelatin for use in an inhaler or insufflator and the like may be formulated containing a powder mix of the compound and a suitable powder base such as lactose or starch.

The compounds may be formulated for parenteral administration by injection, e.g., by bolus injection or continuous infusion. Formulations for injection may be presented in unit-dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active agents may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

Alternatively, the active ingredient may be in powder form for constitution with a suitable vehicle, e.g. sterile pyrogen-free water, before use.

In addition to the formulations described above, the compounds of the present disclosure may also be formulated as a depot preparation. Such long-acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion-exchange resins, or as sparingly soluble derivatives, for example, as a sparingly soluble salt. A pharmaceutical carrier for hydrophobic compounds is a co-solvent system comprising benzyl alcohol, a non-polar surfactant, a water-miscible organic polymer, and an aqueous phase. The co-solvent system may be a VPD co-solvent system. VPD is a solution of 3% w/v benzyl alcohol, 8% w/v of the non-polar surfactant polysorbate 80, and 65% w/v polyethylene glycol 300, made up to volume in absolute ethanol. The VPD co-solvent system (VPD: 5 W) contains VPD diluted 1:1 with a 5% dextrose in water solution. This co-solvent system dissolves hydrophobic compounds well, and itself produces low toxicity upon systemic administration. The proportions of a co-solvent system may be suitably varied without destroying its solubility and toxicity characteristics. Furthermore, the identity of the co-solvent components may be varied: for example, other low-toxicity non-polar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may be substituted for dextrose.

Alternatively, other delivery systems for hydrophobic pharmaceutical compounds may be employed. Liposomes and emulsions are known examples of delivery vehicles or carriers for hydrophobic drugs. Certain organic solvents such as dimethylsulfoxide also may be employed, although usually at the cost of greater toxicity due to the toxic nature of DMSO. Additionally, the compounds may be delivered using a sustained-release system, such as semipermeable matrices of solid hydrophobic polymers containing the therapeutic agent. Various sustained-release materials have been established and are known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid- or gel-phase carriers or excipients. These carriers and excipients may provide marked improvement in the bioavailability of poorly soluble drugs. Examples of such carriers or excipients include calcium carbonate, calcium phosphate, sugars, starches, cellulose derivatives, gelatin, and polymers such as polyethylene glycols. Furthermore, additives or excipients such as Gelucire®, Capryol®, Labrafil®, Labrasol®, Lauroglycol®, Plurol®, Peceol®, Transcutol® and the like may be used.

Further, the pharmaceutical composition may be incorporated into a skin patch for delivery of the drug directly onto the skin.

It will be appreciated that the actual dosages of the agents of this disclosure will vary according to the particular agent being used, the particular composition formulated, the mode of administration, and the particular site, host, and disease being treated. Those skilled in the art using conventional dosage-determination tests in view of the experimental data for a given compound may ascertain optimal dosages for a given set of conditions. For oral administration, an exemplary daily dose generally employed will be from about 0.001 to about 1000 mg/kg of body weight, with courses of treatment repeated at appropriate intervals.

Furthermore, the pharmaceutically acceptable formulations of the present disclosure may contain a compound or compounds of the present disclosure, or a salt or solvate thereof, in an amount of about 10 mg to about 2000 mg, or from about 10 mg to about 1500 mg, or from about 10 mg to about 1000 mg, or from about 10 mg to about 750 mg, or from about 10 mg to about 500 mg, or from about 25 mg to about 500 mg, or from about 50 to about 500 mg, or from about 100 mg to about 500 mg. Furthermore, the pharmaceutically acceptable formulations of the present disclosure may contain a compound of the present disclosure, or a salt or solvate thereof, in an amount of about 50 mg, about 100 mg, about 150 mg, about 200 mg, about 250 mg, about 300 mg, about 350 mg, about 400 mg, about 450 mg, or about 500 mg.

Additionally, the pharmaceutically acceptable formulations of the present disclosure may contain a compound of the present disclosure, or a salt or solvate thereof, in an amount from about 0.5 w/w % to about 95 w/w %, or from about 1 w/w % to about 95 w/w %, or from about 1 w/w % to about 75 w/w %, or from about 5 w/w % to about 75 w/w %, or from about 10 w/w % to about 75 w/w %, or from about 10 w/w % to about 50 w/w %.

The compounds disclosed herein, or salts or solvates thereof, may be administered to a mammal suffering from abnormal cell growth, such as a human, either alone or as part of a pharmaceutically acceptable formulation, once a week, once a day, twice a day, three times a day, or four times a day, or even more frequently.

Those of ordinary skill in the art will understand that with respect to the compounds of the present disclosure, the particular pharmaceutical formulation, the dosage, and the number of doses given per day to a mammal requiring such treatment, are all choices within the knowledge of one of ordinary skill in the art and can be determined without undue experimentation.

Administration of the compounds disclosed herein may be effected by any method that enables delivery of the compounds to the site of action. These methods include oral routes, intraduodenal routes, parenteral injection (including intravenous, subcutaneous, intramuscular, intravascular or infusion), topical, and rectal administration. Bolus doses can be used, or infusions over a period of 1, 2, 3, 4, 5, 10, 15, 20, 30, 60, 90, 120 or more minutes, or any intermediate time period can also be used, as can infusions lasting 3, 4, 5, 6, 7, 8, 9, 10. 12, 14 16, 20, 24 or more hours or lasting for 1-7 days or more. Infusions can be administered by drip, continuous infusion, infusion pump, metering pump, depot formulation, or any other suitable means.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the present disclosure are dictated by and directly dependent on (a) the unique characteristics of the chemotherapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present disclosure.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present disclosure encompasses intra-patient dose-escalation as determined by the skilled artisan. Determining appropriate dosages and regimens for administration of the chemotherapeutic agent are well-known in the relevant art and would be understood to be encompassed by the skilled artisan once provided the teachings disclosed herein.

Some embodiments provide methods of inhibiting ALK, ROS1, TrkA, TrkB, or TrkC activity, or a combination thereof, in a patient, comprising administering to the patient an effective amount of a compound selected from N-[5-(3,5-difluorobenzyl)-1H-indazol-3-yl]-4-(piperazin-1-yl)-2-(tetrahydro-2H-pyran-4-ylamino) benzamide, N-[5-(3,5-difluorobenzyl)-1H-indazol-3-yl]-4-(4-methyl-piperazin-1-yl)-2-(tetrahydro-2H-pyran-4-ylamino) benzamide, and N-[5-(3,5-difluoro-benzenesulfonyl)-1H-indazol-3-yl]-2-((R)-2-methoxy-1-methyl-ethylamino)-4-(4-methyl-piperazin-1-yl) benzamide or a pharmaceutically acceptable salt thereof.

Some embodiments provide methods of inhibiting ALK, ROS1, TrkA, TrkB, or TrkC activity, or a combination thereof, in a patient, comprising administering to the patient an effective amount of a compound which is N-[5-(3,5-difluorobenzyl)-1H-indazol-3-yl]-4-(pip erazin-1-yl)-2-(tetrahydro-2H-pyran-4-ylamino) benzamide or a pharmaceutically acceptable salt thereof.

Some embodiments provide methods of inhibiting ALK, ROS1, TrkA, TrkB, or TrkC activity, or a combination thereof, in a patient, comprising administering to the patient an effective amount of a compound which is N-[5-(3,5-difluorobenzyl)-1H-indazol-3-yl]-4-(4-methyl-piperazin-1-yl)-2-(tetrahydro-2H-pyran-4-ylamino) benzamide, or a pharmaceutically acceptable salt thereof.

Some embodiments provide methods of inhibiting ALK, ROS1, TrkA, TrkB, or TrkC activity, or a combination thereof, in a patient, comprising administering to the patient an effective amount of a compound which is N-[5-(3,5-difluoro-benzenesulfonyl)-1H-indazol-3-yl]-2-((R)-2-methoxy-1-methyl-ethylamino)-4-(4-methyl-piperazin-1-yl) benzamide or a pharmaceutically acceptable salt thereof.

Some embodiments provide methods of treating cancer in a patient in need thereof, the method comprising inhibiting ALK, ROS1, TrkA, TrkB, or TrkC activity, or a combination thereof, in the patient, by administering to the patient an effective amount of a compound selected from N-[5-(3,5-difluorobenzyl)-1 H-indazol-3-yl]-4-(piperazin-1-yl)-2-(tetrahydro-2H-pyran-4-ylamino) benzamide, N-[5-(3,5-difluorobenzyl)-1 H-indazol-3-yl]-4-(4-methyl-pip erazin-1-yl)-2-(tetrahydro-2H-pyran-4-ylamino) benzamide, and N-[5-(3,5-difluoro-benzenesulfonyl)-1H-indazol-3-yl]-2-((R)-2-methoxy-1-methyl-ethylamino)-4-(4-methyl-piperazin-1-yl) benzamide or a pharmaceutically acceptable salt thereof.

Some embodiments provide methods of treating cancer in a patient in need thereof, the method comprising inhibiting ALK, ROS1, TrkA, TrkB, or TrkC activity, or a combination thereof, in the patient, by administering to the patient an effective amount of a compound which is N-[5-(3,5-difluorobenzyl)-1H-indazol-3-yl]-4-(pip erazin-1-yl)-2-(tetrahydro-2H-pyran-4-ylamino) benzamide, or a pharmaceutically acceptable salt thereof.

Some embodiments provide methods of treating cancer in a patient in need thereof, the method comprising inhibiting ALK, ROS1, TrkA, TrkB, or TrkC activity, or a combination thereof, in the patient, by administering to the patient an effective amount of a compound which is N-[5-(3,5-difluorobenzyl)-1H-indazol-3-yl]-4-(4-methyl-pip erazin-1-yl)-2-(tetrahydro-2H-pyran-4-ylamino) benzamide, or a pharmaceutically acceptable salt thereof.

Some embodiments provide methods of treating cancer in a patient in need thereof, the method comprising inhibiting ALK, ROS1, TrkA, TrkB, or TrkC activity, or a combination thereof, in the patient, by administering to the patient an effective amount of a compound which is N-[5-(3,5-difluoro-benzenesulfonyl)-1H-indazol-3-yl]-2-((R)-2-methoxy-1-methyl-ethylamino)-4-(4-methyl-piperazin-1-yl) benzamide or a pharmaceutically acceptable salt thereof.

Some embodiments provide methods of treating non-small cell lung cancer, papillary thyroid cancer, neuroblastoma, pancreatic cancer or colorectal cancer in a patient, comprising administering to the patient an effective amount of a compound selected from N-[5-(3,5-difluorobenzyl)-1H-indazol-3-yl]-4-(piperazin-1-yl)-2-(tetrahydro-2H-pyran-4-ylamino) benzamide, N-[5-(3,5-difluorobenzyl)-1H-indazol-3-yl]-4-(4-methyl-piperazin-1-yl)-2-(tetrahydro-2H-pyran-4-ylamino) benzamide, and N-[5-(3,5-difluoro-benzenesulfonyl)-1H-indazol-3-yl]-2-((R)-2-methoxy-1-methyl-ethylamino)-4-(4-methyl-piperazin-1-yl) benzamide or a pharmaceutically acceptable salt thereof.

Some embodiments provide methods of treating tumors in a patient, the methods comprising administering to the patient an effective amount of a compound selected from N-[5-(3,5-difluorobenzyl)-1H-indazol-3-yl]-4-(piperazin-1-yl)-2-(tetrahydro-2H-pyran-4-ylamino) benzamide, N-[5-(3,5-difluorobenzyl)-1H-indazol-3-yl]-4-(4-methyl-piperazin-1-yl)-2-(tetrahydro-2H-pyran-4-ylamino) benzamide, and N-[5-(3,5-difluoro-benzenesulfonyl)-1H-indazol-3-yl]-2-((R)-2-methoxy-1-methyl-ethylamino)-4-(4-methyl-piperazin-1-yl) benzamide or a pharmaceutically acceptable salt thereof.

Some embodiments provide methods wherein the tumors are caused by the presence of non-small cell lung cancer, papillary thyroid cancer, neuroblastoma, pancreatic cancer or colorectal cancer in the patient. Some embodiments provide methods wherein one or more of the cells comprising the tumors in the patient test positive for the presence of a gene that expresses at least one of ALK, ROS1, TrkA, TrkB, or TrkC kinase or one or more of the cells comprising the tumors in the patient demonstrates at least one of ALK, ROS1, TrkA, TrkB, or TrkC kinase activity.

Some embodiments provide methods wherein one or more of the cells comprising the tumors in the patient test positive for at least one gene rearrangement comprising the gene, or a fragment thereof, that expresses at least one of ALK, ROS1, TrkA, TrkB, or TrkC kinase. Some embodiments provide such methods wherein the cells test positive for at least one of ROS1, TrkA, TrkB, or TrkC kinases. Some embodiments provide methods wherein the cells test positive for ROS1 kinase. Some embodiments provide methods wherein the cells test positive for at least one of TrkA, TrkB and TrkC kinase. Some embodiments provide methods wherein the cells test positive for TrkA kinase. Some embodiments provide methods wherein the cells test positive for TrkB kinase. Some embodiments provide such methods wherein the cells test positive for TrkC kinase.

Some embodiments provide methods of treating cancer in a patient, the method comprising: (1) testing one or more cells comprising the tumors in the patient for the presence of at least one of ALK, ROS1, TrkA, TrkB, or TrkC kinase; and (2) administering to the patient an effective amount of a compound selected from N-[5-(3,5-difluorobenzyl)-1H-indazol-3-yl]-4-(piperazin-1-yl)-2-(tetrahydro-2H-pyran-4-ylamino) benzamide, N-[5-(3,5-difluorobenzyl)-1H-indazol-3-yl]-4-(4-methyl-piperazin-1-yl)-2-(tetrahydro-2H-pyran-4-ylamino) benzamide, and N-[5-(3,5-difluoro-benzenesulfonyl)-1H-indazol-3-yl]-2-((R)-2-methoxy-1-methyl-ethylamino)-4-(4-methyl-piperazin-1-yl) benzamide, or a pharmaceutically acceptable salt thereof, if the one or more cells tests positive for at least one of ALK, ROS1, TrkA, TrkB, or TrkC kinase.

Some embodiments provide methods of treating cancer in a patient, the method comprising: (1) testing one or more cells comprising the tumors in the patient for the presence of at least one of ROS1, TrkA, TrkB, or TrkC kinase; and (2) administering to the patient an effective amount of a compound selected from N-[5-(3,5-difluorobenzyl)-1H-indazol-3-yl]-4-(piperazin-1-yl)-2-(tetrahydro-2H-pyran-4-ylamino) benzamide, N-[5-(3,5-difluorobenzyl)-1H-indazol-3-yl]-4-(4-methyl-piperazin-1-yl)-2-(tetrahydro-2H-pyran-4-ylamino) benzamide, and N-[5-(3,5-difluoro-benzenesulfonyl)-1H-indazol-3-yl]-2-((R)-2-methoxy-1-methyl-ethylamino)-4-(4-methyl-piperazin-1-yl) benzamide, or a pharmaceutically acceptable salt thereof, if the one or more cells tests positive for at least one of ROS1, TrkA, TrkB, or TrkC kinase.

Some embodiments provide methods wherein the patient is administered an effective amount of N-[5-(3,5-difluorobenzyl)-1H-indazol-3-yl]-4-(piperazin-1-yl)-2-(tetrahydro-2H-pyran-4-ylamino) benzamide, or a pharmaceutically acceptable salt thereof. Some embodiments provide methods wherein the patient is administered an effective amount of N-[5-(3,5-difluorobenzyl)-1H-indazol-3-yl]-4-(4-methyl-piperazin-1-yl)-2-(tetrahydro-2H-pyran-4-ylamino) benzamide, or a pharmaceutically acceptable salt thereof. Some embodiments provide methods wherein the patient is administered an effective amount of N-[5-(3,5-difluoro-benzenesulfonyl)-1H-indazol-3-yl]-2-((R)-2-methoxy-1-methyl-ethylamino)-4-(4-methyl-piperazin-1-yl) benzamide, or a pharmaceutically acceptable salt thereof.

Some embodiments provide a method of treating a cancer patient, comprising (a) acquiring knowledge of the presence of at least one genetic alteration in at least one target gene in the cancer patient, wherein the at least one target gene is selected from ALK1, BDNF, NGF, NGFR, NTF3, NTF4, ROS1, SORT1, NTRK1, NTRK2, and NTRK3; (b) selecting a compound selected from N-[5-(3,5-difluorobenzyl)-1H-indazol-3-yl]-4-(piperazin-1-yl)-2-(tetrahydro-2H-pyran-4-ylamino) benzamide, N-[5-(3,5-difluorobenzyl)-1H-indazol-3-yl]-4-(4-methyl-piperazin-1-yl)-2-(tetrahydro-2H-pyran-4-ylamino) benzamide, and N-[5-(3,5-difluoro-benzenesulfonyl)-1H-indazol-3-yl]-2-((R)-2-methoxy-1-methyl-ethylamino)-4-(4-methyl-piperazin-1-yl) benzamide, or a pharmaceutically acceptable salt thereof, as a treatment for the cancer patient, based on the recognition that the compound is effective in treating cancer patients having the at least one genetic alteration in the at least one target gene; and (c) administering a therapeutically effective amount of the compound to the cancer patient.

Some embodiments provide a method of treating a cancer patient, comprising administering to the cancer patient a therapeutically effective amount of a compound selected from N-[5-(3,5-difluorobenzyl)-1H-indazol-3-yl]-4-(piperazin-1-yl)-2-(tetrahydro-2H-pyran-4-ylamino) benzamide, N-[5-(3,5-difluorobenzyl)-1H-indazol-3-yl]-4-(4-methyl-piperazin-1-yl)-2-(tetrahydro-2H-pyran-4-ylamino) benzamide, and N-[5-(3,5-difluoro-benzenesulfonyl)-1H-indazol-3-yl]-2-((R)-2-methoxy-1-methyl-ethylamino)-4-(4-methyl-piperazin-1-yl) benzamide, or a pharmaceutically acceptable salt thereof, wherein prior to the administration of the compound, the cancer patient is known to possess at least one genetic alteration in at least one target gene selected from ALK1, BDNF, NGF, NGFR, NTF3, NTF4, ROS1, SORT1, NTRK1, NTRK2, and NTRK3.

Some embodiments provide a method of treating cancer in a patient, comprising administering to the cancer patient known to possess at least one genetic alteration in at least one target gene selected from ALK1, BDNF, NGF, NGFR, NTF3, NTF4, ROS1, SORT1, NTRK1, NTRK2, and NTRK3 a therapeutically effective amount of a compound selected from N-[5-(3,5-difluorobenzyl)-1H-indazol-3-yl]-4-(piperazin-1-yl)-2-(tetrahydro-2H-pyran-4-ylamino) benzamide, N-[5-(3,5-difluorobenzyl)-1H-indazol-3-yl]-4-(4-methyl-piperazin-1-yl)-2-(tetrahydro-2H-pyran-4-ylamino) benzamide, and N-[5-(3,5-difluoro-benzenesulfonyl)-1H-indazol-3-yl]-2-((R)-2-methoxy-1-methyl-ethylamino)-4-(4-methyl-piperazin-1-yl) benzamide, or a pharmaceutically acceptable salt thereof.

Some embodiments provide a method of treating a cancer patient, wherein the cancer patient is known to possess at least one genetic alteration in at least one target gene, comprising administering to the cancer patient a therapeutically effective amount of a compound selected from N-[5-(3,5-difluorobenzyl)-1H-indazol-3-yl]-4-(piperazin-1-yl)-2-(tetrahydro-2H-pyran-4-ylamino) benzamide, N-[5-(3,5-difluorobenzyl)-1H-indazol-3-yl]-4-(4-methyl-piperazin-1-yl)-2-(tetrahydro-2H-pyran-4-ylamino) benzamide, and N-[5-(3,5-difluoro-benzenesulfonyl)-1H-indazol-3-yl]-2-((R)-2-methoxy-1-methyl-ethylamino)-4-(4-methyl-piperazin-1-yl) benzamide, or a pharmaceutically acceptable salt thereof, and wherein the target gene is selected from ALK1, BDNF, NGF, NGFR, NTF3, NTF4, ROS1, SORT1, NTRK1, NTRK2, and NTRK3.

Some embodiments provide a method of treating a cancer patient, wherein prior to the treatment the patient is known to possess at least one genetic alteration in at least one target gene, comprising administering to the cancer patient a therapeutically effective amount of a compound selected from N-[5-(3,5-difluorobenzyl)-1H-indazol-3-yl]-4-(piperazin-1-yl)-2-(tetrahydro-2H-pyran-4-ylamino) benzamide, N-[5-(3,5-difluorobenzyl)-1H-indazol-3-yl]-4-(4-methyl-piperazin-1-yl)-2-(tetrahydro-2H-pyran-4-ylamino) benzamide, and N-[5-(3,5-difluoro-benzenesulfonyl)-1H-indazol-3-yl]-2-((R)-2-methoxy-1-methyl-ethylamino)-4-(4-methyl-piperazin-1-yl) benzamide, or a pharmaceutically acceptable salt thereof, and wherein the target gene is selected from ALK1, BDNF, NGF, NGFR, NTF3, NTF4, ROS1, SORT1, NTRK1, NTRK2, and NTRK3.

Some embodiments provide a method of treating a cancer patient, comprising administering to the cancer patient a therapeutically effective amount of a compound selected from N-[5-(3,5-difluorobenzyl)-1H-indazol-3-yl]-4-(piperazin-1-yl)-2-(tetrahydro-2H-pyran-4-ylamino) benzamide, N-[5-(3,5-difluorobenzyl)-1H-indazol-3-yl]-4-(4-methyl-piperazin-1-yl)-2-(tetrahydro-2H-pyran-4-ylamino) benzamide, and N-[5-(3,5-difluoro-benzenesulfonyl)-1H-indazol-3-yl]-2-((R)-2-methoxy-1-methyl-ethylamino)-4-(4-methyl-piperazin-1-yl) benzamide, or a pharmaceutically acceptable salt thereof, and wherein prior to the compound being administered to the patient, the patient is known to possess at least one genetic alteration in at least one target gene selected from ALK1, BDNF, NGF, NGFR, NTF3, NTF4, ROS1, SORT1, NTRK1, NTRK2, and NTRK3.

Some embodiments provide a method for treating a cancer patient, comprising (a) acquiring knowledge of the presence of at least one genetic alteration in at least one target gene selected from ALK1, BDNF, NGF, NGFR, NTF3, NTF4, ROS1, SORT1, NTRK1, NTRK2, and NTRK3; and (b) administering to the patient a therapeutically effective amount of a compound selected from N-[5-(3,5-difluorobenzyl)-1H-indazol-3-yl]-4-(piperazin-1-yl)-2-(tetrahydro-2H-pyran-4-ylamino) benzamide, N-[5-(3,5-difluorobenzyl)-1H-indazol-3-yl]-4-(4-methyl-piperazin-1-yl)-2-(tetrahydro-2H-pyran-4-ylamino) benzamide, and N-[5-(3,5-difluoro-benzenesulfonyl)-1H-indazol-3-yl]-2-((R)-2-methoxy-1-methyl-ethylamino)-4-(4-methyl-piperazin-1-yl) benzamide, or a pharmaceutically acceptable salt thereof.

Some embodiments provide any of the methods described herein wherein the patient or subject is suffering from cancer and the cancer is selected from at least one of non-small cell lung cancer, papillary thyroid cancer, neuroblastoma, pancreatic cancer and colorectal cancer.

Some embodiments provide a pharmaceutical composition comprising a compound disclosed herein in combination with one or more chemotherapeutic agents or radiotherapy, such as radiotherapy as commonly administered to treat, ameliorate the symptoms of, or prevent or delay the onset of cancer. Such agents can include, but are not limited to, antihormonal agents such as antiestrogens, antiandrogens and aromatase inhibitors, topoisomerase I inhibitors, topoisomerase II inhibitors, agents that target microtubules, platin-based agents, alkylating agents, DNA damaging or intercalating agents, antineoplastic antimetabolites, other kinase inhibitors, other anti-angiogenic agents, inhibitors of kinesins, therapeutic monoclonal antibodies, inhibitors of mTOR, histone deacetylase inhibitors, farnesyl transferase inhibitors, and inhibitors of hypoxic response.

Some embodiments provide a product or kit comprising a compound disclosed herein or a pharmaceutically acceptable salt thereof, as defined above, or pharmaceutical compositions thereof and one or more chemotherapeutic agents, as a combined preparation for simultaneous, separate or sequential use in anticancer therapy. Some embodiments provide a product or kit comprising a compound selected from N-[5-(3,5-difluorobenzyl)-1H-indazol-3-yl]-4-(piperazin-1-yl)-2-(tetrahydro-2H-pyran-4-ylamino) benzamide and N-[5-(3,5-difluorobenzyl)-1H-indazol-3-yl]-4-(4-methyl-piperazin-1-yl)-2-(tetrahydro-2H-pyran-4-ylamino) benzamide, or a pharmaceutically acceptable salt thereof, or pharmaceutical compositions thereof and one or more chemotherapeutic agents, as a combined preparation for simultaneous, separate or sequential use in anticancer therapy.

Some embodiments provide a product or kit comprising a compound which is N-[5-(3,5-difluorobenzyl)-1H-indazol-3-yl]-4-(piperazin-1-yl)-2-(tetrahydro-2H-pyran-4-ylamino) benzamide, or a pharmaceutically acceptable salt thereof, or pharmaceutical compositions thereof and one or more chemotherapeutic agents, as a combined preparation for simultaneous, separate or sequential use in anticancer therapy. Some embodiments provide a product or kit comprising a compound which is N-[5-(3,5-difluorobenzyl)-1H-indazol-3-yl]-4-(4-methyl-piperazin-1-yl)-2-(tetrahydro-2H-pyran-4-ylamino) benzamide, or a pharmaceutically acceptable salt thereof, or pharmaceutical compositions thereof and one or more chemotherapeutic agents, as a combined preparation for simultaneous, separate or sequential use in anticancer therapy.

Some embodiments provide a compound disclosed herein a pharmaceutically acceptable salt thereof, as defined above, for use as a medicament.

Some embodiments provide the use of the compounds disclosed herein or a pharmaceutically acceptable salt thereof, as defined above, in the manufacture of a medicament with antitumor activity.

Some embodiments include any of the methods described herein, wherein the cancer is selected from non-small cell lung cancer, papillary thyroid cancer, neuroblastoma, pancreatic cancer and colorectal cancer. Some embodiments are any of the methods described herein wherein the cancer is non-small cell lung cancer. Some embodiments include any of the methods described herein, wherein the cancer is the cancer is papillary thyroid cancer. Some embodiments include any of the methods described herein, wherein the cancer is wherein the cancer is neuroblastoma. Some embodiments include any of the methods described herein, wherein the cancer is wherein the cancer is pancreatic cancer. Some embodiments include any of the methods described herein, wherein the cancer is wherein the cancer is colorectal cancer.

The compounds of the present disclosure may be formulated into pharmaceutical compositions as described below in any pharmaceutical form recognizable to the skilled artisan as being suitable. Pharmaceutical compositions of the present disclosure comprise a therapeutically effective amount of at least one compound disclosed herein and an inert, pharmaceutically acceptable carrier or diluent.

To treat or prevent diseases or conditions mediated by ALK, ROS1, TrkA, TrkB, or TrkC, or a combination thereof, a pharmaceutical composition of the present disclosure is administered in a suitable formulation prepared by combining a therapeutically effective amount (i.e., a ALK, ROS1, TrkA, TrkB, or TrkC modulating, regulating, or inhibiting amount effective to achieve therapeutic efficacy) of at least one compound of the present disclosure (as an active ingredient) with one or more pharmaceutically suitable carriers, which may be selected, for example, from diluents, excipients and auxiliaries that facilitate processing of the active compounds into the final pharmaceutical preparations.

The pharmaceutical carriers employed may be either solid or liquid. Exemplary solid carriers are lactose, sucrose, talc, gelatin, agar, pectin, acacia, magnesium stearate, stearic acid and the like. Exemplary liquid carriers are syrup, peanut oil, olive oil, water and the like. Similarly, the inventive compositions may include time-delay or time-release material known in the art, such as glyceryl monostearate or glyceryl distearate alone or with a wax, ethylcellulose, hydroxypropylmethylcellulose, methylmethacrylate or the like. Further additives or excipients may be added to achieve the desired formulation properties. For example, a bioavailability enhancer, such as Labrasol, Gelucire or the like, or formulator, such as CMC (carboxy-methylcellulose), PG (propyleneglycol), or PEG (polyethyleneglycol), may be added. Gelucire®, a semi-solid vehicle that protects active ingredients from light, moisture and oxidation, may be added, e.g., when preparing a capsule formulation.

If a solid carrier is used, the preparation can be tableted, placed in a hard gelatin capsule in powder or pellet form, or formed into a troche or lozenge. The amount of solid carrier may vary, but generally will be from about 25 mg to about 1 g. If a liquid carrier is used, the preparation may be in the form of syrup, emulsion, soft gelatin capsule, sterile injectable solution or suspension in an ampoule or vial or non-aqueous liquid suspension. If a semi-solid carrier is used, the preparation may be in the form of hard and soft gelatin capsule formulations. The inventive compositions are prepared in unit-dosage form appropriate for the mode of administration, e.g. parenteral or oral administration.

To obtain a stable water-soluble dose form, a salt of a compound of the present disclosure may be dissolved in an aqueous solution of an organic or inorganic acid, such as a 0.3 M solution of succinic acid or citric acid. If a soluble salt form is not available, the agent may be dissolved in a suitable co-solvent or combinations of co-solvents. Examples of suitable co-solvents include alcohol, propylene glycol, polyethylene glycol 300, polysorbate 80, glycerin and the like in concentrations ranging from 0 to 60% of the total volume. In an exemplary embodiment, a compound of the present disclosure is dissolved in DMSO and diluted with water. The composition may also be in the form of a solution of a salt form of the active ingredient in an appropriate aqueous vehicle such as water or isotonic saline or dextrose solution.

Proper formulation is dependent upon the route of administration selected. For injection, the agents of the compounds of the present disclosure may be formulated into aqueous solutions, preferably in physiologically compatible buffers such as Hanks solution, Ringer's solution, or physiological saline buffer. For transmucosal administration, penetrants appropriate to the barrier to be permeated are used in the formulation. Such penetrants are generally known in the art.

For oral administration, the compounds can be formulated by combining the active compounds with pharmaceutically acceptable carriers known in the art. Such carriers enable the compounds of the present disclosure to be formulated as tablets, pills, dragees, capsules, liquids, gels, syrups, slurries, suspensions and the like, for oral ingestion by a subject to be treated. Pharmaceutical preparations for oral use can be obtained using a solid excipient in admixture with the active ingredient (agent), optionally grinding the resulting mixture, and processing the mixture of granules after adding suitable auxiliaries, if desired, to obtain tablets or dragee cores. Suitable excipients include: fillers such as sugars, including lactose, sucrose, mannitol, or sorbitol; and cellulose preparations, for example, maize starch, wheat starch, rice starch, potato starch, gelatin, gum, methyl cellulose, hydroxypropylmethyl-cellulose, sodium carboxymethylcellulose, or polyvinylpyrrolidone (PVP). If desired, disintegrating agents may be added, such as crosslinked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active agents.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate, and, optionally, stabilizers. In soft capsules, the active agents may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

For administration intranasally or by inhalation, the compounds for use according to the present disclosure may be conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of gelatin for use in an inhaler or insufflator and the like may be formulated containing a powder mix of the compound and a suitable powder base such as lactose or starch.

The compounds may be formulated for parenteral administration by injection, e.g., by bolus injection or continuous infusion. Formulations for injection may be presented in unit-dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active agents may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

Alternatively, the active ingredient may be in powder form for constitution with a suitable vehicle, e.g. sterile pyrogen-free water, before use.

In addition to the formulations described above, the compounds of the present disclosure may also be formulated as a depot preparation. Such long-acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion-exchange resins, or as sparingly soluble derivatives, for example, as a sparingly soluble salt. A pharmaceutical carrier for hydrophobic compounds is a co-solvent system comprising benzyl alcohol, a non-polar surfactant, a water-miscible organic polymer, and an aqueous phase. The co-solvent system may be a VPD co-solvent system. VPD is a solution of 3% w/v benzyl alcohol, 8% w/v of the non-polar surfactant polysorbate 80, and 65% w/v polyethylene glycol 300, made up to volume in absolute ethanol. The VPD co-solvent system (VPD: 5 W) contains VPD diluted 1:1 with a 5% dextrose in water solution. This co-solvent system dissolves hydrophobic compounds well, and itself produces low toxicity upon systemic administration. The proportions of a co-solvent system may be suitably varied without destroying its solubility and toxicity characteristics. Furthermore, the identity of the co-solvent components may be varied: for example, other low-toxicity non-polar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may be substituted for dextrose.

Alternatively, other delivery systems for hydrophobic pharmaceutical compounds may be employed. Liposomes and emulsions are known examples of delivery vehicles or carriers for hydrophobic drugs. Certain organic solvents such as dimethylsulfoxide also may be employed, although usually at the cost of greater toxicity due to the toxic nature of DMSO. Additionally, the compounds may be delivered using a sustained-release system, such as semipermeable matrices of solid hydrophobic polymers containing the therapeutic agent. Various sustained-release materials have been established and are known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid- or gel-phase carriers or excipients. These carriers and excipients may provide marked improvement in the bioavailability of poorly soluble drugs. Examples of such carriers or excipients include calcium carbonate, calcium phosphate, sugars, starches, cellulose derivatives, gelatin, and polymers such as polyethylene glycols. Furthermore, additives or excipients such as Gelucire®, Capryol®, Labrafil®, Labrasol®, Lauroglycol®, Plurol®, Peceol®, Transcutol® and the like may be used.

Further, the pharmaceutical composition may be incorporated into a skin patch for delivery of the drug directly onto the skin.

It will be appreciated that the actual dosages of the agents of this disclosure will vary according to the particular agent being used, the particular composition formulated, the mode of administration, and the particular site, host, and disease being treated. Those skilled in the art using conventional dosage-determination tests in view of the experimental data for a given compound may ascertain optimal dosages for a given set of conditions. For oral administration, an exemplary daily dose generally employed will be from about 0.001 to about 1000 mg/kg of body weight, with courses of treatment repeated at appropriate intervals.

Furthermore, the pharmaceutically acceptable formulations of the present disclosure may contain a compound of the present disclosure, or a salt or solvate thereof, in an amount of about 10 mg to about 2000 mg, or from about 10 mg to about 1500 mg, or from about 10 mg to about 1000 mg, or from about 10 mg to about 750 mg, or from about 10 mg to about 500 mg, or from about 25 mg to about 500 mg, or from about 50 to about 500 mg, or from about 100 mg to about 500 mg. Furthermore, the pharmaceutically acceptable formulations of the present disclosure may contain a compound of the present disclosure, or a salt or solvate thereof, in an amount of about 50 mg, about 100 mg, about 150 mg, about 200 mg, about 250 mg, about 300 mg, about 350 mg, about 400 mg, about 450 mg, or about 500 mg.

Additionally, the pharmaceutically acceptable formulations of the present disclosure may contain a compound of the present disclosure, or a salt or solvate thereof, in an amount from about 0.5 w/w % to about 95 w/w %, or from about 1 w/w % to about 95 w/w %, or from about 1 w/w % to about 75 w/w %, or from about 5 w/w % to about 75 w/w %, or from about 10 w/w % to about 75 w/w %, or from about 10 w/w % to about 50 w/w %.

The compounds of the present disclosure, or salts or solvates thereof, may be administered to a mammal suffering from abnormal cell growth, such as a human, either alone or as part of a pharmaceutically acceptable formulation, once a day, twice a day, three times a day, or four times a day, or even more frequently.

Those of ordinary skill in the art will understand that with respect to the compounds of the present disclosure, the particular pharmaceutical formulation, the dosage, and the number of doses given per day to a mammal requiring such treatment, are all choices within the knowledge of one of ordinary skill in the art and can be determined without undue experimentation.

Administration of the compounds disclosed herein may be effected by any method that enables delivery of the compounds to the site of action. These methods include oral routes, intraduodenal routes, parenteral injection (including intravenous, subcutaneous, intramuscular, intravascular or infusion), topical, and rectal administration.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the present disclosure are dictated by and directly dependent on (a) the unique characteristics of the chemotherapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present disclosure.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present disclosure encompasses intra-patient dose-escalation as determined by the skilled artisan. Determining appropriate dosages and regimens for administration of the chemotherapeutic agent are well-known in the relevant art and would be understood to be encompassed by the skilled artisan once provided the teachings disclosed herein.

The compounds, compositions and methods provided herein are useful for the treatment of cancers including but not limited to cancers of the: circulatory system, for example, heart (sarcoma [angiosarcoma, fibrosarcoma, rhabdomyosarcoma, liposarcoma], myxoma, rhabdomyoma, fibroma, lipoma and teratoma), mediastinum and pleura, and other intrathoracic organs, vascular tumors and tumor-associated vascular tissue; respiratory tract, for example, nasal cavity and middle ear, accessory sinuses, larynx, trachea, bronchus and lung such as small cell lung cancer (SCLC), non-small cell lung cancer (NSCLC), bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; gastrointestinal system, for example, esophagus (squamous cell carcinoma, adenocarcinoma, leiomyosarcoma, lymphoma), stomach (carcinoma, lymphoma, leiomyosarcoma), gastric, pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); genitourinary tract, for example, kidney (adenocarcinoma, Wilm's tumor [nephroblastoma], lymphoma, leukemia), bladder and/or urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); liver, for example, hepatoma (hepatocellular carcinoma), cholangiocarcinoma, hepatoblastoma, angiosarcoma, hepatocellular adenoma, hemangioma, pancreatic endocrine tumors (such as pheochromocytoma, insulinoma, vasoactive intestinal peptide tumor, islet cell tumor and glucagonoma); bone, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; nervous system, for example, neoplasms of the central nervous system (CNS), primary CNS lymphoma, skull cancer (osteoma, hemangioma, granuloma, xanthoma, osteitis deformans), meninges (meningioma, meningiosarcoma, gliomatosis), brain cancer (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); reproductive system, for example, gynecological, uterus (endometrial carcinoma), cervix (cervical carcinoma, pre- tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma) and other sites associated with female genital organs; placenta, penis, prostate, testis, and other sites associated with male genital organs; hematologic system, for example, blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; oral cavity, for example, lip, tongue, gum, floor of mouth, palate, and other parts of mouth, parotid gland, and other parts of the salivary glands, tonsil, oropharynx, nasopharynx, pyriform sinus, hypopharynx, and other sites in the lip, oral cavity and pharynx; skin, for example, malignant melanoma, cutaneous melanoma, basal cell carcinoma, squamous cell carcinoma, Karposi's sarcoma, moles dysplastic nevi, lipoma, angioma, dermatofibroma, and keloids; adrenal glands: neuroblastoma; and other tissues including connective and soft tissue, retroperitoneum and peritoneum, eye, intraocular melanoma, and adnexa, breast, head or/and neck, anal region, thyroid, parathyroid, adrenal gland and other endocrine glands and related structures, secondary and unspecified malignant neoplasm of lymph nodes, secondary malignant neoplasm of respiratory and digestive systems and secondary malignant neoplasm of other sites.

More specifically, examples of “cancer” when used herein in connection with the present disclosure include cancer selected from lung cancer (NSCLC and SCLC), cancer of the head or neck, ovarian cancer, colon cancer, rectal cancer, prostate cancer, cancer of the anal region, stomachcancer, breast cancer, cancer of the kidney or ureter, renal cell carcinoma, carcinoma of the renal pelvis, neoplasms of the central nervous system (CNS), primary CNS lymphoma, non-Hodgkins's lymphoma, spinal axis tumors, or a combination of one or more of the foregoing cancers.

In some embodiments, the compounds and the compositions disclosed herein are useful for the treatment of cancers, including Spitz melanoma, perineural invasion, pulmonary large cell neuroendocrine carcinoma, uterine carcinoma, juvenile breast cancer, nasopharyngeal carcinoma, adenoid cystic cancer, meduallary thyroid cancer, salivary cancer, congenital infantile fibrosarcoma, mesoblastic nephroma, esophageal cancer (squamous), diffuse large B-cell lymphoma, papillary thyroid cancer, and mammary analogue secretory carcinoma.

In some embodiments, the compounds disclosed herein may be used in combination with one or more additional anti-cancer agents which are described below. When a combination therapy is used, the one or more additional anti-cancer agents may be administered sequentially or simultaneously with the compound of the disclosure. In some embodiments, the additional anti-cancer agent is administered to a mammal (e.g., a human) prior to administration of the compound of the disclosure. In some embodiments, the additional anti-cancer agent is administered to the mammal after administration of the compound of the disclosure. In some embodiments, the additional anti-cancer agent is administered to the mammal (e.g., a human) simultaneously with the administration of the compound disclosed herein.

Some embodiments also relate to a pharmaceutical composition for the treatment of abnormal cell growth in a mammal, including a human, which comprises an amount of a compound disclosed herein, as defined above (including hydrates, solvates and polymorphs of the compound or pharmaceutically acceptable salts thereof), in combination with one or more (preferably one to three) anti-cancer agents selected from the group consisting of anti-angiogenesis agents and signal transduction inhibitors and a pharmaceutically acceptable carrier, wherein the amounts of the active agent and the combination anti-cancer agents when taken as a whole is therapeutically effective for treating the abnormal cell growth.

In some embodiments, the anti-cancer agent used in conjunction with a compound disclosed herein and pharmaceutical compositions described herein is an anti-angiogenesis agent (e.g., an agent that stops tumors from developing new blood vessels). Examples of anti-angiogenesis agents include for example VEGF inhibitors, VEGFR inhibitors, TIE-2 inhibitors, PDGFR inhibitors, angiopoetin inhibitors, PKC.beta. inhibitors, COX-2 (cyclooxygenase II) inhibitors, integrins (alpha-v/beta-3), MMP-2 (matrix-metalloproteinase 2) inhibitors, and MMP-9 (matrix-metalloproteinase 9) inhibitors. Preferred anti-angiogenesis agents include sunitinib (Sutent®), bevacizumab (Avastin®), axitinib (AG 13736), SU 14813 (Pfizer), and AG 13958 (Pfizer).

Additional anti-angiogenesis agents include vatalanib (CGP 79787), Sorafenib (Nexavar®), pegaptanib octasodium (Macugen®), vandetanib (Zactima®), PF-0337210 (Pfizer), SU 14843 (Pfizer), AZD 2171 (AstraZeneca), ranibizumab (Lucentis®), Neovastat® (AE 941), tetrathiomolybdata (Coprexa®), AMG 706 (Amgen), VEGF Trap (AVE 0005), CEP 7055 (Sanofi-Aventis), XL 880 (Exelixis), telatinib (BAY 57-9352), and CP-868,596 (Pfizer).

Other anti-angiogenesis agents include enzastaurin (LY 317615), midostaurin (CGP 41251), perifosine (KRX 0401), teprenone (Selbex®) and UCN 01 (Kyowa Hakko).

Other examples of anti-angiogenesis agents which can be used in conjunction with a compound of Disclosed herein and pharmaceutical compositions described herein include celecoxib (Celebrex®), parecoxib (Dynastat®), deracoxib (SC 59046), lumiracoxib (Preige®), valdecoxib (Bextra®), rofecoxib (Vioxx®), iguratimod (Careram®), IP 751 (Invedus), SC-58125 (Pharmacia) and etoricoxib (Arcoxia®).

Other anti-angiogenesis agents include exisulind (Aptosyn®), salsalate (Amigesic®), diflunisal (Dolobid®), ibuprofen (Motrin®), ketoprofen (Orudis®) nabumetone (Relafen®), piroxicam (Feldene®), naproxen (Aleve®, Naprosyn®) diclofenac (Voltaren®), indomethacin (Indocin®), sulindac (Clinoril®), tolmetin (Tolectin®), etodolac (Lodine®), ketorolac (Toradol®), and oxaprozin (Daypro®).

Other anti-angiogenesis agents include ABT 510 (Abbott), apratastat (TMI 005), AZD 8955 (AstraZeneca), incyclinide (Metastat®), and PCK 3145 (Procyon).

Other anti-angiogenesis agents include acitretin (Neotigason®), plitidepsin (Aplidine®), cilengtide (EMD 121974), combretastatin A4 (CA4P), fenretinide (4 HPR), halofuginone (Tempostatin®), Panzem® (2-methoxyestradiol), PF-03446962 (Pfizer), rebimastat (BMS 275291), catumaxomab (Removab®), lenalidomide (Revlimid®) squalamine (EVIZON®), thalidomide (Thalomid®), Ukrain® (NSC 631570), Vitaxin® (MEDI 522), and zoledronic acid (Zometa®).

In some embodiments, the anti-cancer agent is a so called signal transduction inhibitor (e.g., inhibiting the means by which regulatory molecules that govern the fundamental processes of cell growth, differentiation, and survival communicated within the cell). Signal transduction inhibitors include small molecules, antibodies, and antisense molecules. Signal transduction inhibitors include for example kinase inhibitors (e.g., tyrosine kinase inhibitors or serine/threonine kinase inhibitors) and cell cycle inhibitors. More specifically signal transduction inhibitors include, for example, ALK inhibitors, ROS1 inhibitors, TrkA inhibitors, TrkB inhibitors, TrkC inhibitors, farnesyl protein transferase inhibitors, EGF inhibitor, ErbB-1 (EGFR), ErbB-2, pan erb, IGF1R inhibitors, MEK, c-Kit inhibitors, FLT-3 inhibitors, K-Ras inhibitors, PI3 kinase inhibitors, JAK inhibitors, STAT inhibitors, Raf kinase inhibitors, Akt inhibitors, mTOR inhibitor, P70S6 kinase inhibitors, inhibitors of the WNT pathway and so called multi-targeted kinase inhibitors.

Preferred signal transduction inhibitors include gefitinib (Iressa®), cetuximab (Erbitux®), erlotinib (Tarceva®), trastuzumab (Herceptin®), sunitinib (Sutent®) imatinib (Gleevec®), and PD325901 (Pfizer).

Additional examples of signal transduction inhibitors which may be used in conjunction with a compound of Disclosed herein and pharmaceutical compositions described herein include BMS 214662 (Bristol-Myers Squibb), lonafarnib (Sarasar®), pelitrexol (AG 2037), matuzumab (EMD 7200), nimotuzumab (TheraClM h-R3®), panitumumab (Vectibix®), Vandetanib (Zactima®), pazopanib (SB 786034), ALT 110 (Alteris Therapeutics), BIBW 2992 (Boehringer Ingelheim), and Cervene® (TP 38).

Other examples of signal transduction inhibitor include PF-2341066 (Pfizer), PF-299804 (Pfizer), canertinib (CI 1033), pertuzumab (Omnitarg®), Lapatinib (Tycerb®), pelitinib (EKB 569), miltefosine (Miltefosin®), BMS 599626 (Bristol-Myers Squibb), Lapuleucel-T (Neuvenge®), NeuVax® (E75 cancer vaccine), Osidem® (IDM 1), mubritinib (TAK-165), CP-724,714 (Pfizer), panitumumab (Vectibix®), lapatinib (Tycerb®), PF-299804 (Pfizer), pelitinib (EKB 569), and pertuzumab (Omnitarg®).

Other examples of signal transduction inhibitors include ARRY 142886 (Array Biopharm), everolimus (Certican®), zotarolimus (Endeavor®), temsirolimus (Torisel®), AP 23573 (ARIAD), and VX 680 (Vertex).

Additionally, other signal transduction inhibitors include XL 647 (Exelixis), sorafenib (Nexavar®), LE-AON (Georgetown University), and GI-4000 (GlobeImmune).

Other signal transduction inhibitors include ABT 751 (Abbott), alvocidib (flavopiridol), BMS 387032 (Bristol Myers), EM 1421 (Erimos), indisulam (E 7070), seliciclib (CYC 200), BIO 112 (One Bio), BMS 387032 (Bristol-Myers Squibb), PD 0332991 (Pfizer), AG 024322 (Pfizer), LOXO-101 (Loxo Oncology), crizotinib, and ceritinib.

In some embodiments, the compounds of disclosed herein are used together with classical antineoplastic agents. Classical antineoplastic agents include but are not limited to hormonal modulators such as hormonal, anti-hormonal, androgen agonist, androgen antagonist and anti-estrogen therapeutic agents, histone deacetylase (HDAC) inhibitors, gene silencing agents or gene activating agents, ribonucleases, proteosomics, Topoisomerase I inhibitors, Camptothecin derivatives, Topoisomerase II inhibitors, alkylating agents, antimetabolites, poly(ADP-ribose) polymerase-1 (PARP-1) inhibitor, microtubulin inhibitors, antibiotics, plant derived spindle inhibitors, platinum-coordinated compounds, gene therapeutic agents, antisense oligonucleotides, vascular targeting agents (VTAs), and statins.

Examples of classical antineoplastic agents used in combination therapy with a compound of disclosed herein, optionally with one or more other agents include, but are not limited to, glucocorticoids, such as dexamethasone, prednisone, prednisolone, methylprednisolone, hydrocortisone, and progestins such as medroxyprogesterone, megestrol acetate (Megace), mifepristone (RU-486), Selective Estrogen Receptor Modulators (SERMs; such as tamoxifen, raloxifene, lasofoxifene, afimoxifene, arzoxifene, bazedoxifene, fispemifene, ormeloxifene, ospemifene, tesmilifene, toremifene, trilostane and CHF 4227 (Cheisi)), Selective Estrogen-Receptor Downregulators (SERD's; such as fulvestrant), exemestane (Aromasin), anastrozole (Arimidex), atamestane, fadrozole, letrozole (Femara), gonadotropin-releasing hormone (GnRH; also commonly referred to as luteinizing hormone-releasing hormone [LHRH]) agonists such as buserelin (Suprefact), goserelin (Zoladex), leuprorelin (Lupron), and triptorelin (Trelstar), abarelix (Plenaxis), bicalutamide (Casodex), cyproterone, flutamide (Eulexin), megestrol, nilutamide (Nilandron), and osaterone, dutasteride, epristeride, finasteride, Serenoa repens, PHL 00801, abarelix, goserelin, leuprorelin, triptorelin, bicalutamide, tamoxifen, exemestane, anastrozole, fadrozole, formestane, letrozole, and combinations thereof.

Other examples of classical antineoplastic agents used in combination with compounds of disclosed herein include but are not limited to suberolanilide hydroxamic acid (SAHA, Merck Inc./Aton Pharmaceuticals), depsipeptide (FR901228 or FK228), G2M-777, MS-275, pivaloyloxymethyl butyrate and PXD-101; Onconase (ranpirnase), PS-341 (MLN-341), Velcade (bortezomib), 9-aminocamptothecin, belotecan, BN-80915 (Roche), camptothecin, diflomotecan, edotecarin, exatecan (Daiichi), gimatecan, 10-hydroxycamptothecin, irinotecan HCl (Camptosar), lurtotecan, Orathecin (rubitecan, Supergen), SN-38, topotecan, camptothecin, 10-hydroxycamptothecin, 9-aminocamptothecin, irinotecan, SN-38, edotecarin, topotecan, aclarubicin, adriamycin, amonafide, amrubicin, annamycin, daunorubicin, doxorubicin, elsamitrucin, epirubicin, etoposide, idarubicin, galarubicin, hydroxycarbamide, nemorubicin, novantrone (mitoxantrone), pirarubicin, pixantrone, procarbazine, rebeccamycin, sobuzoxane, tafluposide, valrubicin, Zinecard (dexrazoxane), nitrogen mustard N-oxide, cyclophosphamide, AMD-473, altretamine, AP-5280, apaziquone, brostallicin, bendamustine, busulfan, carboquone, carmustine, chlorambucil, dacarbazine, estramustine, fotemustine, glufosfamide, ifosfamide, KW-2170, lomustine, mafosfamide, mechlorethamine, melphalan, mitobronitol, mitolactol, mitomycin C, mitoxatrone, nimustine, ranimustine, temozolomide, thiotepa, and platinum-coordinated alkylating compounds such as cisplatin, Paraplatin (carboplatin), eptaplatin, lobaplatin, nedaplatin, Eloxatin (oxaliplatin, Sanofi), streptozocin, satrplatin, and combinations thereof.

In some embodiments, the compounds of disclosed herein are used together with dihydrofolate reductase inhibitors (such as methotrexate and NeuTrexin (trimetresate glucuronate)), purine antagonists (such as 6-mercaptopurine riboside, mercaptopurine, 6-thioguanine, cladribine, clofarabine (Clolar), fludarabine, nelarabine, and raltitrexed), pyrimidine antagonists (such as 5-fluorouracil (5-FU), Alimta (premetrexed disodium, LY231514, MTA), capecitabine (Xeloda®), cytosine arabinoside, Gemzar® (gemcitabine, Eli Lilly), Tegafur (UFT Orzel or Uforal and including TS-1 combination of tegafur, gimestat and otostat), doxifluridine, carmofur, cytarabine (including ocfosfate, phosphate stearate, sustained release and liposomal forms), enocitabine, 5-azacitidine (Vidaza), decitabine, and ethynylcytidine) and other antimetabolites such as eflornithine, hydroxyurea, leucovorin, nolatrexed (Thymitaq), triapine, trimetrexate, N-(5-[N-(3,4-dihydro-2-methyl-4-oxoquinazolin-6-ylmethyl)-N-methylamino]-2-thenyl)-L-glutamic acid, AG-014699 (Pfizer Inc.), ABT-472 (Abbott Laboratories), INO-1001 (Inotek Pharmaceuticals), KU-0687 (KuDOS Pharmaceuticals) and GPI 18180 (Guilford Pharm Inc) and combinations thereof.

Other examples of classical antineoplastic cytotoxic agents used in combination therapy with a compound of disclosed herein, optionally with one or more other agents include, but are not limited to, Abraxane (Abraxis BioScience, Inc.), Batabulin (Amgen), EPO 906 (Novartis), Vinflunine (Bristol-Myers Squibb Company), actinomycin D, bleomycin, mitomycin C, neocarzinostatin (Zinostatin), vinblastine, vincristine, vindesine, vinorelbine (Navelbine), docetaxel (Taxotere), Ortataxel, paclitaxel (including Taxoprexin a DHA/paciltaxel conjugate), cisplatin, carboplatin, Nedaplatin, oxaliplatin (Eloxatin), Satraplatin, Camptosar, capecitabine (Xeloda), oxaliplatin (Eloxatin), Taxotere alitretinoin, Canfosfamide (Telcyta®), DMXAA (Antisoma), ibandronic acid, L-asparaginase, pegaspargase (Oncaspar®), Efaproxiral (Efaproxyn®—radiation therapy)), bexarotene (Targretin®), Tesmilifene (DPPE—enhances efficacy of cytotoxics)), Theratope® (Biomira), Tretinoin (Vesanoid®), tirapazamine (Trizaone®), motexafin gadolinium (Xcytrin®) Cotara® (mAb), and NBI-3001 (Protox Therapeutics), polyglutamate-paclitaxel (Xyotax®) and combinations thereof.

Further examples of classical antineoplastic agents used in combination therapy with a compound of disclosed herein, optionally with one or more other agents include, but are not limited to, as Advexin (ING 201), TNFerade (GeneVec, a compound which express TNFalpha in response to radiotherapy), RB94 (Baylor College of Medicine), Genasense (Oblimersen, Genta), Combretastatin A4P (CA4P), Oxi-4503, AVE-8062, ZD-6126, TZT-1027, Atorvastatin (Lipitor, Pfizer Inc.), Provastatin (Pravachol, Bristol-Myers Squibb), Lovastatin (Mevacor, Merck Inc.), Simvastatin (Zocor, Merck Inc.), Fluvastatin (Lescol, Novartis), Cerivastatin (Baycol, Bayer), Rosuvastatin (Crestor, AstraZeneca), Lovostatin, Niacin (Advicor, Kos Pharmaceuticals), Caduet, Lipitor, torcetrapib, and combinations thereof.

Some embodiments relate to a method for the treatment of breast cancer in a human in need of such treatment, comprising administering to the human an amount of a compound of disclosed herein, in combination with one or more (preferably one to three) anti-cancer agents selected from the group consisting of trastuzumab, tamoxifen, docetaxel, paclitaxel, capecitabine, gemcitabine, vinorelbine, exemestane, letrozole and anastrozole.

Some embodiments provide a method of treating colorectal cancer in a mammal, such as a human, in need of such treatment, by administering an amount of a compound of disclosed herein, in combination with one or more (preferably one to three) anti-cancer agents. Examples of particular anti-cancer agents include those typically used in adjuvant chemotherapy, such as FOLFOX, a combination of 5-fluorouracil (5-FU) or capecitabine (Xeloda), leucovorin and oxaliplatin (Eloxatin). Further examples of particular anti-cancer agents include those typically used in chemotherapy for metastatic disease, such as FOLFOX or FOLFOX in combination with bevacizumab (Avastin); and FOLFIRI, a combination of 5-FU or capecitabine, leucovorin and irinotecan (Camptosar). Further examples include 17-DMAG, ABX-EFR, AMG-706, AMT-2003, ANX-510 (CoFactor), aplidine (plitidepsin, Aplidin), Aroplatin, axitinib (AG-13736), AZD-0530, AZD-2171, bacillus Calmette-Guerin (BCG), bevacizumab (Avastin), BIO-117, BIO-145, BMS-184476, BMS-275183, BMS-528664, bortezomib (Velcade), C-1311 (Symadex), cantuzumab mertansine, capecitabine (Xeloda), cetuximab (Erbitux), clofarabine (Clofarex), CMD-193, combretastatin, Cotara, CT-2106, CV-247, decitabine (Dacogen), E-7070, E-7820, edotecarin, EMD-273066, enzastaurin (LY-317615) epothilone B (EPO-906), erlotinib (Tarceva), flavopyridol, GCAN-101, gefitinib (Iressa), huA33, huC242-DM4, imatinib (Gleevec), indisulam, ING-1, irinotecan (CPT-11, Camptosar) ISIS 2503, ixabepilone, lapatinib (Tykerb), mapatumumab (HGS-ETR1), MBT-0206, MEDI-522 (Abregrin), Mitomycin, MK-0457 (VX-680), MLN-8054, NB-1011, NGR-TNF, NV-1020, oblimersen (Genasense, G3139), OncoVex, ONYX 015 (CI-1042), oxaliplatin (Eloxatin), panitumumab (ABX-EGF, Vectibix), pelitinib (EKB-569), pemetrexed (Alimta), PD-325901, PF-0337210, PF-2341066, RAD-001 (Everolimus), RAV-12, Resveratrol, Rexin-G, S-1 (TS-1), seliciclib, SN-38 liposome, Sodium stibogluconate (SSG), sorafenib (Nexavar), SU-14813, sunitinib (Sutent), temsirolimus (CCI 779), tetrathiomolybdate, thalomide, TLK-286 (Telcyta), topotecan (Hycamtin), trabectedin (Yondelis), vatalanib (PTK-787), vorinostat (SAHA, Zolinza), WX-UK1, and ZYC300, wherein the amounts of the active agent together with the amounts of the combination anticancer agents are effective in treating colorectal cancer.

Some embodiments provide methods for the treatment of renal cell carcinoma in a human in need of such treatment, comprising administering to the human an amount of a compound of disclosed herein, in combination with one or more (preferably one to three) anti-cancer agents selected from the group consisting of capecitabine (Xeloda), interferon alpha, interleukin-2, bevacizumab (Avastin), gemcitabine (Gemzar), thalidomide, cetuximab (Erbitux), vatalanib (PTK-787), Sutent, AG-13736, SU-11248, Tarceva, Iressa, Lapatinib and Gleevec, wherein the amounts of the active agent together with the amounts of the combination anticancer agents is effective in treating renal cell carcinoma.

Some embodiments provide methods for the treatment of melanoma in a human in need of such treatment, comprising administering to the human an amount of a compound of disclosed herein, in combination with one or more (preferably one to three) anti-cancer agents selected from the group consisting of interferon alpha, interleukin-2, temozolomide (Temodar), docetaxel (Taxotere), paclitaxel, Dacarbazine (DTIC), carmustine (also known as BCNU), Cisplatin, vinblastine, tamoxifen, PD-325,901, Axitinib, bevacizumab (Avastin), thalidomide, sorafanib, vatalanib (PTK-787), Sutent, CpG-7909, AG-13736, Iressa, Lapatinib and Gleevec, wherein the amounts of the active agent together with the amounts of the combination anticancer agents is effective in treating melanoma.

Some embodiments provide methods for the treatment of lung cancer in a human in need of such treatment, comprising administering to the human an amount of a compound disclosed herein, in combination with one or more (preferably one to three) anti-cancer agents selected from the group consisting of capecitabine (Xeloda), bevacizumab (Avastin), gemcitabine (Gemzar), docetaxel (Taxotere), paclitaxel, premetrexed disodium (Alimta), Tarceva, Iressa, Vinorelbine, Irinotecan, Etoposide, Vinblastine, and Paraplatin (carboplatin), wherein the amounts of the active agent together with the amounts of the combination anticancer agents is effective in treating lung cancer.

In some embodiments are provided methods for treating neuroblastoma in a patient, the method comprising administering to the patient a therapeutically effective amount of at least one compound selected from N-[5-(3,5-difluorobenzyl)-1H-indazol-3-yl]-4-(piperazin-1-yl)-2-(tetrahydro-2H-pyran-4-ylamino) benzamide, N-[5-(3,5-difluorobenzyl)-1H-indazol-3-yl]-4-(4-methyl-piperazin-1-yl)-2-(tetrahydro-2H-pyran-4-ylamino) benzamide, and N-[5-(3,5-difluoro-benzenesulfonyl)-1H-indazol-3-yl]-2-((R)-2-methoxy-1-methyl-ethylamino)-4-(4-methyl-piperazin-1-yl) benzamide, or a pharmaceutically acceptable salt thereof, in combination with at least one chemotherapeutic agent. In some embodiments the neuroblastoma is tropomyosin-receptor-kinase positive. In some embodiments are provided such methods, wherein the compound administered to the patient in combination with at least one chemotherapeutic agent is N-[5-(3,5-difluorobenzyl)-1H-indazol-3-yl]-4-(piperazin-1-yl)-2-(tetrahydro-2H-pyran-4-ylamino) benzamide, or a pharmaceutically acceptable salt thereof. In some embodiments are provided such methods, wherein the compound administered to the patient in combination with at least one chemotherapeutic agent is N-[5-(3,5-difluorobenzyl)-1H-indazol-3-yl]-4-(4-methyl- piperazin-1-yl)-2-(tetrahydro-2H-pyran-4-ylamino) benzamide, or a pharmaceutically acceptable salt thereof. In some embodiments are provided such methods, wherein the compound administered to the patient in combination with at least one chemotherapeutic agent is N-[5-(3,5-difluoro-benzenesulfonyl)-1H-indazol-3-yl]-2-((R)-2-methoxy-1-methyl-ethylamino)-4-(4-methyl-piperazin-1-yl) benzamide, or a pharmaceutically acceptable salt thereof. In some embodiments are provided such methods, wherein the at least one chemotherapeutic agent comprises at least one topoisomerase I inhibitor. In some embodiments are provided such methods, wherein the at least one chemotherapeutic agent comprises at least one alkylating agent. In some embodiments are provided such methods, wherein the at least one chemotherapeutic agent comprises at least one topoisomerase I inhibitor and at least one alkylating agent. In some embodiments are provided such methods, wherein the at least one topoisomerase I inhibitor is irinotecan. In some embodiments are provided such methods, wherein the at least one alkylating agent is temozolomide. In some embodiments are provided such methods, wherein the tropomyosin-receptor-kinase is at least one of TrkA, TrkB, and TrkC. In some embodiments are provided such methods, wherein the tropomyosin-receptor-kinase is TrkA. In some embodiments are provided such methods, wherein the tropomyosin-receptor-kinase is TrkB. In some embodiments are provided such methods, wherein the tropomyosin-receptor-kinase is TrkC. In some embodiments are provided such methods, wherein the tropomyosin-receptor-kinases are TrkA and TrkB. In some embodiments are provided such methods, wherein the tropomyosin-receptor-kinases are TrkA and TrkC. In some embodiments are provided such methods, wherein the tropomyosin-receptor-kinases are TrkB and TrkC. In some embodiments are provided such methods, wherein the tropomyosin-receptor-kinases are TrkA and TrkB and TrkC.

In some embodiments are provided methods for treating neuroblastoma in a patient, wherein the neuroblastoma is tropomyosin-receptor-kinase positive, comprising administering to the patient a therapeutically effective amount of at least one compound selected from N-[5-(3,5-difluorobenzyl)-1H-indazol-3-yl]-4-(piperazin-1-yl)-2-(tetrahydro-2H-pyran-4-ylamino) benzamide, N-[5-(3,5-difluorobenzyl)-1H-indazol-3-yl]-4-(4-methyl-piperazin-1-yl)-2-(tetrahydro-2H-pyran-4-ylamino) benzamide, and N-[5-(3,5-difluoro-benzenesulfonyl)-1H-indazol-3-yl]-2-((R)-2-methoxy-1-methyl-ethylamino)-4-(4-methyl-piperazin-1-yl) benzamide, or a pharmaceutically acceptable salt thereof, in combination with at least one chemotherapeutic agent. In some embodiments are provided such methods, wherein the compound administered to the patient in combination with at least one chemotherapeutic agent is N-[5-(3,5-difluorobenzyl)-1H-indazol-3-yl]-4-(piperazin-1-yl)-2-(tetrahydro-2H-pyran-4-ylamino) benzamide, or a pharmaceutically acceptable salt thereof. In some embodiments are provided such methods, wherein the compound administered to the patient in combination with at least one chemotherapeutic agent is N-[5-(3,5-difluorobenzyl)-1H-indazol-3-yl]-4-(4-methyl-piperazin-1-yl)-2-(tetrahydro-2H-pyran-4-ylamino) benzamide, or a pharmaceutically acceptable salt thereof. In some embodiments are provided such methods, wherein the compound administered to the patient in combination with at least one chemotherapeutic agent is N-[5-(3,5-difluoro-benzenesulfonyl)-1H-indazol-3-yl]-2-((R)-2-methoxy-1-methyl-ethylamino)-4-(4-methyl-piperazin-1-yl) benzamide, or a pharmaceutically acceptable salt thereof. In some embodiments are provided such methods, wherein the at least one chemotherapeutic agent comprises at least one topoisomerase I inhibitor. In some embodiments are provided such methods, wherein the at least one chemotherapeutic agent comprises at least one alkylating agent. In some embodiments are provided such methods, wherein the at least one chemotherapeutic agent comprises at least one topoisomerase I inhibitor and at least one alkylating agent. In some embodiments are provided such methods, wherein the at least one topoisomerase I inhibitor is irinotecan. In some embodiments are provided such methods, wherein the at least one alkylating agent is temozolomide. In some embodiments are provided such methods, wherein the tropomyosin-receptor-kinase is at least one of TrkA, TrkB, and TrkC. In some embodiments are provided such methods, wherein the tropomyosin-receptor-kinase is TrkA. In some embodiments are provided such methods, wherein the tropomyosin-receptor-kinase is TrkB. In some embodiments are provided such methods, wherein the tropomyosin-receptor-kinase is TrkC.

In some embodiments are provided methods for treating neuroblastoma in a patient, wherein the neuroblastoma is tropomyosin-receptor-kinase positive, comprising administering to the patient a therapeutically effective amount of at least one compound selected from N-[5-(3,5-difluorobenzyl)-1H-indazol-3-yl]-4-(piperazin-1-yl)-2-(tetrahydro-2H-pyran-4-ylamino) benzamide, N-[5-(3,5-difluorobenzyl)-1H-indazol-3-yl]-4-(4-methyl-piperazin-1-yl)-2-(tetrahydro-2H-pyran-4-ylamino) benzamide, and N-[5-(3,5-difluoro-benzenesulfonyl)-1H-indazol-3-yl]-2-((R)-2-methoxy-1-methyl-ethylamino)-4-(4-methyl-piperazin-1-yl) benzamide, or a pharmaceutically acceptable salt thereof, in combination with at least one chemotherapeutic agent. In some embodiments are provided such methods, wherein the compound administered to the patient in combination with at least one chemotherapeutic agent is N-[5-(3,5-difluorobenzyl)-1H-indazol-3-yl]-4-(piperazin-1-yl)-2-(tetrahydro-2H-pyran-4-ylamino) benzamide, or a pharmaceutically acceptable salt thereof. In some embodiments are provided such methods, wherein the compound administered to the patient in combination with at least one chemotherapeutic agent is N-[5-(3,5-difluorobenzyl)-1H-indazol-3-yl]-4-(4-methyl-piperazin-1-yl)-2-(tetrahydro-2H-pyran-4-ylamino) benzamide, or a pharmaceutically acceptable salt thereof. In some embodiments are provided such methods, wherein the compound administered to the patient in combination with at least one chemotherapeutic agent is N-[5-(3,5-difluoro-benzenesulfonyl)-1H-indazol-3-yl]-2-((R)-2-methoxy-1-methyl-ethylamino)-4-(4-methyl-piperazin-1-yl) benzamide, or a pharmaceutically acceptable salt thereof. In some embodiments are provided such methods, wherein the at least one chemotherapeutic agent comprises at least one topoisomerase I inhibitor. In some embodiments are provided such methods, wherein the at least one chemotherapeutic agent comprises at least one alkylating agent. In some embodiments are provided such methods, wherein the at least one chemotherapeutic agent comprises at least one topoisomerase I inhibitor and at least one alkylating agent. In some embodiments are provided such methods, wherein the at least one topoisomerase I inhibitor is irinotecan. In some embodiments are provided such methods, wherein the at least one alkylating agent is temozolomide. In some embodiments are provided such methods, wherein the tropomyosin-receptor-kinase is at least one of TrkA, TrkB, and TrkC. In some embodiments are provided such methods, wherein the tropomyosin-receptor-kinase is TrkA. In some embodiments are provided such methods, wherein the tropomyosin-receptor-kinase is TrkB. In some embodiments are provided such methods, wherein the tropomyosin-receptor-kinase is TrkC.

In some embodiments are provided methods for treating neuroblastoma in a patient, the method comprising: (1) testing one or more cells comprising the neuroblastoma in the patient for the presence of tropomyosin-receptor-kinase; and (2) administering to the patient an effective amount of at least one compound selected from N-[5-(3,5-difluorobenzyl)-1H-indazol-3-yl]-4-(piperazin-1-yl)-2-(tetrahydro-2H-pyran-4-ylamino) benzamide, N-[5-(3,5-difluorobenzyl)-1H-indazol-3-yl]-4-(4-methyl-piperazin-1-yl)-2-(tetrahydro-2H-pyran-4-ylamino) benzamide, and N-[5-(3,5-difluoro-benzenesulfonyl)-1H-indazol-3-yl]-2-((R)-2-methoxy-1-methyl-ethylamino)-4-(4-methyl-piperazin-1-yl) benzamide, or a pharmaceutically acceptable salt thereof, in combination with at least one chemotherapeutic agent if the one or more cells tests positive for tropomyosin-receptor-kinase. In some embodiments are provided such methods, wherein the compound administered to the patient in combination with at least one chemotherapeutic agent is N-[5-(3,5-difluorobenzyl)-1H-indazol-3-yl]-4-(piperazin-1-yl)-2-(tetrahydro-2H-pyran-4-ylamino) benzamide, or a pharmaceutically acceptable salt thereof. In some embodiments are provided such methods, wherein the compound administered to the patient in combination with at least one chemotherapeutic agent is N-[5-(3,5-difluorobenzyl)-1H-indazol-3-yl]-4-(4-methyl-piperazin-1-yl)-2-(tetrahydro-2H-pyran-4-ylamino) benzamide, or a pharmaceutically acceptable salt thereof. In some embodiments are provided such methods, wherein the compound administered to the patient in combination with at least one chemotherapeutic agent is N-[5-(3,5-difluoro-benzenesulfonyl)-1H-indazol-3-yl]-2-((R)-2-methoxy-1-methyl-ethylamino)-4-(4-methyl-piperazin-1-yl) benzamide, or a pharmaceutically acceptable salt thereof. In some embodiments are provided such methods, wherein the at least one chemotherapeutic agent comprises at least one topoisomerase I inhibitor. In some embodiments are provided such methods, wherein the at least one chemotherapeutic agent comprises at least one alkylating agent. In some embodiments are provided such methods, wherein the at least one chemotherapeutic agent comprises at least one topoisomerase I inhibitor and at least one alkylating agent. In some embodiments are provided such methods, wherein the at least one topoisomerase I inhibitor is irinotecan. In some embodiments are provided such methods, wherein the at least one alkylating agent is temozolomide. In some embodiments are provided such methods, wherein the tropomyosin-receptor-kinase is at least one of TrkA, TrkB, and TrkC. In some embodiments are provided such methods, wherein the tropomyosin-receptor-kinase is TrkA. In some embodiments are provided such methods, wherein the tropomyosin-receptor-kinase is TrkB. In some embodiments are provided such methods, wherein the tropomyosin-receptor-kinase is TrkC. In some embodiments are provided such methods, wherein the tropomyosin-receptor-kinases are TrkA and TrkB. In some embodiments are provided such methods, wherein the tropomyosin-receptor-kinases are TrkA and TrkC. In some embodiments are provided such methods, wherein the tropomyosin-receptor-kinases are TrkB and TrkC. In some embodiments are provided such methods, wherein the tropomyosin-receptor-kinases are TrkA and TrkB and TrkC.

In some embodiments are provided methods for treating tropomyosin-receptor-kinase positive neuroblastoma in a patient, comprising administering to the patient a therapeutically effective amount of at least one compound selected from N-[5-(3,5-difluorobenzyl)-1H-indazol-3-yl]-4-(piperazin-1-yl)-2-(tetrahydro-2H-pyran-4-ylamino) benzamide, N-[5-(3,5-difluorobenzyl)-1 H-indazol-3-yl]-4-(4-methyl-piperazin-1-yl)-2-(tetrahydro-2H-pyran-4-ylamino) benzamide, and N-[5-(3,5-difluoro-benzenesulfonyl)-1H-indazol-3-yl]-2-((R)-2-methoxy-1-methyl-ethylamino)-4-(4-methyl-piperazin-1-yl) benzamide, or a pharmaceutically acceptable salt thereof, in combination with at least one chemotherapeutic agent. In some embodiments are provided such methods, wherein the compound administered to the patient in combination with at least one chemotherapeutic agent is N-[5-(3,5-difluorobenzyl)-1H-indazol-3-yl]-4-(piperazin-1-yl)-2-(tetrahydro-2H-pyran-4-ylamino) benzamide, or a pharmaceutically acceptable salt thereof. In some embodiments are provided such methods, wherein the compound administered to the patient in combination with at least one chemotherapeutic agent is N-[5-(3,5-difluorobenzyl)-1H-indazol-3-yl]-4-(4-methyl-piperazin-1-yl)-2-(tetrahydro-2H-pyran-4-ylamino) benzamide, or a pharmaceutically acceptable salt thereof. In some embodiments are provided such methods, wherein the compound administered to the patient in combination with at least one chemotherapeutic agent is N-[5-(3,5-difluoro-benzenesulfonyl)-1H-indazol-3-yl]-2-((R)-2-methoxy-1-methyl-ethylamino)-4-(4-methyl-piperazin-1-yl) benzamide, or a pharmaceutically acceptable salt thereof. In some embodiments are provided such methods, wherein the at least one chemotherapeutic agent comprises at least one topoisomerase I inhibitor. In some embodiments are provided such methods, wherein the at least one chemotherapeutic agent comprises at least one alkylating agent. In some embodiments are provided such methods, wherein the at least one chemotherapeutic agent comprises at least one topoisomerase I inhibitor and at least one alkylating agent. In some embodiments are provided such methods, wherein the at least one topoisomerase I inhibitor is irinotecan. In some embodiments are provided such methods, wherein the at least one alkylating agent is temozolomide. In some embodiments are provided such methods, wherein the tropomyosin-receptor-kinase is at least one of TrkA, TrkB, and TrkC. In some embodiments are provided such methods, wherein the tropomyosin-receptor-kinase is TrkA. In some embodiments are provided such methods, wherein the tropomyosin-receptor-kinase is TrkB. In some embodiments are provided such methods, wherein the tropomyosin-receptor-kinase is TrkC. In some embodiments are provided such methods, wherein the at least one tropomyosin-receptor-kinase are TrkA and TrkB. In some embodiments are provided such methods, wherein the at least one tropomyosin-receptor-kinase are TrkA and TrkC. In some embodiments are provided such methods, wherein the at least one tropomyosin-receptor-kinase are TrkB and TrkC. In some embodiments are provided such methods, wherein the at least one tropomyosin-receptor-kinase are TrkA and TrkB and TrkC.

Some embodiments disclosed herein relate to methods for selecting a cancer patient who is predicted to respond to the administration of a therapeutic regimen, including (a) acquiring knowledge of the presence of one or more molecular alterations in a biological sample from the cancer patient, wherein the one or more molecular alterations is detected by an assay including one or more antibodies that bind to one or more of ALK, ROS1, TrkA, TrkB, and TrkC biomarkers; and (b) selecting the patient as predicted to respond to the administration of a therapeutic regimen if the one or more molecular alterations is detected in one or more of the biomarkers, or selecting the patient as predicted to not respond to the administration of a therapeutic regimen if the one or more molecular alterations is not detected in the biomarkers. In such methods, the therapeutic regiment includes administering to the selected patient a therapeutically effective amount of one or more chemotherapeutic agents selected from N-[5-(3,5-difluorobenzyl)-1H-indazol-3-yl]-4-(piperazin-1-yl)-2-(tetrahydro-2H-pyran-4-ylamino) benzamide, N-[5-(3,5-difluorobenzyl)-1H-indazol-3-yl]-4-(4-methyl-piperazin-1-yl)-2-(tetrahydro-2H-pyran-4-ylamino) benzamide, and N-[5-(3,5-difluoro-benzenesulfonyl)-1H-indazol-3-yl]-2-((R)-2-methoxy-1-methyl-ethylamino)-4-(4-methyl-piperazin-1-yl) benzamide, or a pharmaceutically acceptable salt thereof. In some embodiments are provided such methods, wherein the selected chemotherapeutic agent is N-[5-(3,5-difluorobenzyl)-1H-indazol-3-yl]-4-(piperazin-1-yl)-2-(tetrahydro-2H-pyran-4-ylamino) benzamide, or a pharmaceutically acceptable salt thereof. In some embodiments are provided such methods, wherein the selected chemotherapeutic agent is N-[5-(3,5-difluorobenzyl)-1H-indazol-3-yl]-4-(4-methyl-piperazin-1-yl)-2-(tetrahydro-2H-pyran-4-ylamino) benzamide, or a pharmaceutically acceptable salt thereof. In some embodiments are provided such methods, wherein the selected chemotherapeutic agent is N-[5-(3,5-difluoro-benzenesulfonyl)-1H-indazol-3-yl]-2- ((R)-2-methoxy-1-methyl-ethylamino)-4-(4-methyl-piperazin-1-yl) benzamide, or a pharmaceutically acceptable thereof.

The discussion of the general methods given herein is intended for illustrative purposes only. Other alternative methods and alternatives will be apparent to those of skill in the art upon review of this disclosure, and are to be included within the spirit and purview of this application.

### EXAMPLES

Applicants have developed immunohistochemistry (IHC) reagents comprising mixtures of antibodies that bind to various proteins, for example antibodies that bind to ALK, ROS1, TrkA, TrkB and TrkC, for use in simultaneously monitoring levels of tyrosine kinase ALK, ROS1, and TrkA, TrkB, TrkC proteins in research and clinical applications such as, for example, in methods for screening large populations of cancer patients for prevalence studies and/or for chemotherapeutic treatments as disclosed herein.

Additional alternatives are disclosed in further detail in the following examples, which are not in any way intended to limit the scope of the claims.

### Example 1

### Simultaneous Detection of Multiple Biomarkers with an Array-Based Protein-Antibody Binding Assay

The goal in this example was to test the feasibility of simultaneously and specifically detecting and/or quantitating expression levels of several biomarkers using immunohistochemistry (IHC) coupled with enhanced diaminobenzidine (DAB) without the use of any expensive machinery. Applicants demonstrated the potential use of this assay to detect and quantitate expression levels of several biomarkers simultaneously in a general laboratory setting.

The feasibility of this assay was first tested by a simplified procedure. The experiments were performed on tissue sections from the following control cell lines:

1) Karpas299 (ALK+, TrkA overexpression);

2) HCC78 (ROS 1+);

3) KM12 (ALK+, TrkA+);

4) BaF3/NTRK2:ETV6 (TrkB+); and

BaF3/NTRK3:ETV6 (TrkC+).

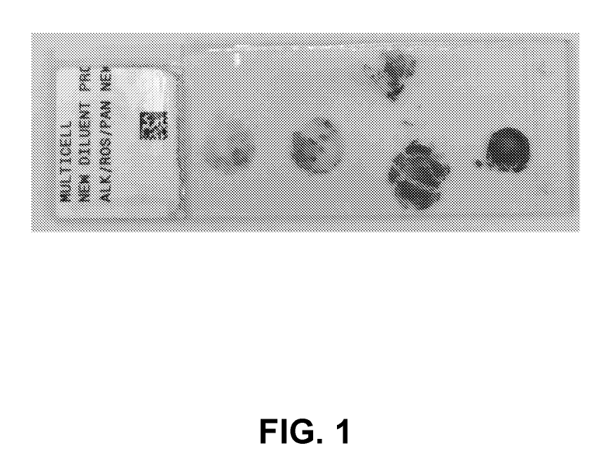

The tissue sections were spotted on a glass slide. To demonstrate that this assay can detect different biomarkers at the same time, the whole glass slide was incubated with individual antibodies or combinations of different antibodies. As shown in FIG. 1, multiple biomarkers could be detected in a protein-array format simultaneously.

A general multiplexed immunohistochemistry (IHC) procedure was developed, where the step of antigen retrieval was performed in EDTA (pH 9) at 98° C. for 30 min. Blocking step was performed using H₂O₂ followed by UV Block (UltraVision Block, Thermo Scientific). When desired, a signal amplification step was performed to enhance sensitivity and visualization of potentially low or weakly expressing cells in addition to the secondary antibody conjugated to a dextran-HRP polymer by using the Envision FLEX system (Dako, Carpinteria, Calif.) in accordance with the manufacturer instructions. Staining signals were developed using diaminobenzidine (DAB). Mayer's Hematoxlyin solution (Sigma Aldrich, St. Louis, Mo.) was used as counter stain.

In a typical multiplexed IHC assay, the mixture of antibodies included the following antibodies (this mixture of antibodies that are selected from bind to one or more of ALK, ROS1, TrkA, TrkB and TrkC may be referred to herein as the “antibody cocktail” or the “pan-IHC cocktail”).

1) anti-ALK monoclonal antibody D5F3®, (Cell Signaling Technologies; Beverly, Mass) 1:500 dilution;

2) anti-ROS1 monoclonal antibody D4D6® (Cell Signaling Technologies; Beverly, Mass), 1:100 dilution; and

3) anti Trk (pan) antibody C17F1® (Cell Signaling Technologies; Beverly, Mass), 1:25 dilution. This antibody has been previously reported to detect all three kinases TrkA, TrkB and TrkC.

FIG. 1 summarizes the results of a multiplexed IHC experiment performed with a mixture of antibodies comprising all three antibodies D5F3®, D4D5®, C17F1®. Positive staining was observed for all five samples derived from the control cell lines Karpas299, HCC78, KM12, BaF3/NTRK2:ETV6, and BaF3/NTRK3:ETV6.

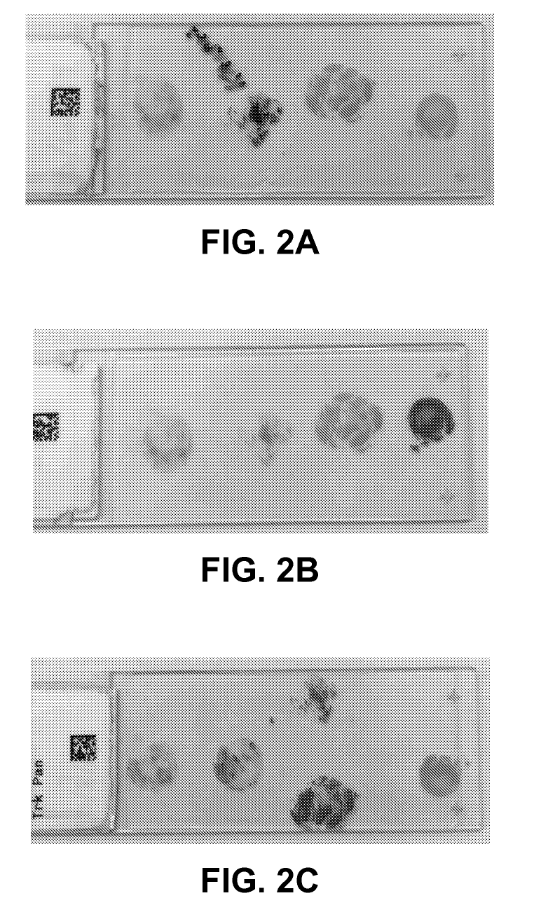

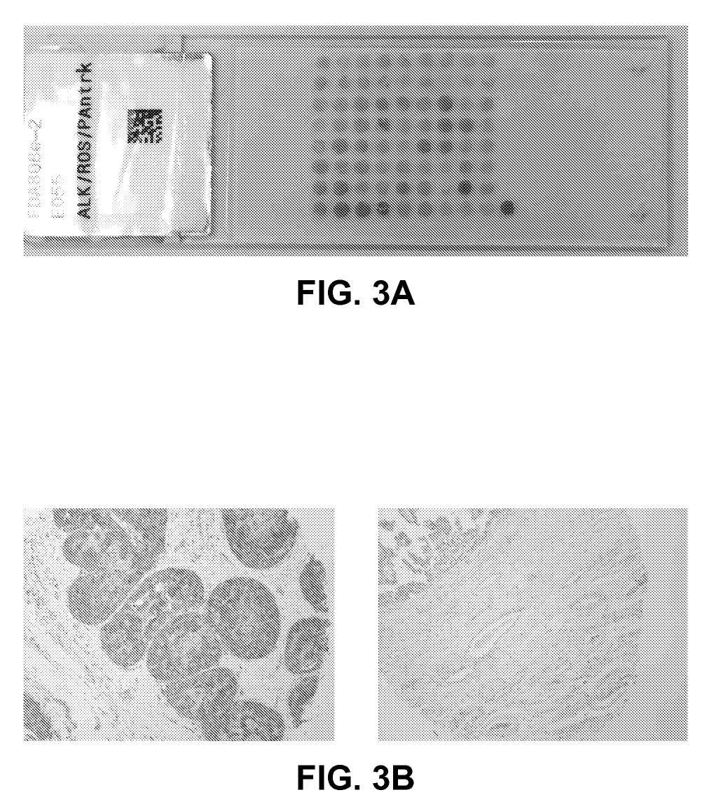

As controls, each of the monoclonal antibodies D5F3®, D4D5®, C17F1® was also tested individually by using the general procedure described above. The results are summarized in FIGS. 2A to 2C. In this experiment, the following control cell lines were included Karpas299, HCC78, KM12, BaF3/NTRK2:ETV6, and BaF3/NTRK3:ETV6. FIG. 2A illustrates the results of an IHC assay performed with anti-ALK antibody D5F3®. As expected, the positive staining was observed in the spot corresponding to the sample derived from Karpas299 (ALK+, TrkA overexpression), thus confirming the specificity of this antibody. FIG. 2B summarizes the results of an IHC assay performed with anti-ROS1 antibody D4D5®. As expected, the positive staining was observed in the spot corresponding to the sample derived from HCC78 (ROS1+; dark spot, far right), thus confirming the specificity of this antibody. The remaining samples were derived from the cell lines Karpas299, BaF3/NTRK2:ETV6, and KM12, and were shown negative. Similarly, FIG. 2C depicts the results of an IHC assay performed with anti-Trk3(pan) antibody C17F1®. Positive staining was observed in the spots corresponding to the samples derived from BaF3/NTRK2:ETV6, BaF3/NTRK3:ETV6, KM12, and Karpas299. This result is expected because both KM12 and Karpas cell lines were known to express TrkA.

### Example 2

### Performance of Multiplexed IHC Assays Performed on Tissue Microarray (TMA)

To assess the performance of the methods described herein, multiplexed immunohistochemistry experiments performed on sections from five following control cell lines: Karpas299 (ALK+, TrkA overexpression); HCC78 (ROS1+); KM12 (ALK+, TrkA+); BaF3/NTRK2:ETV6 (TrkB+); and BaF3/NTRK3:ETV6 (TrkC+). Multiplexed immunohistochemistry was performed on 72 samples derived from various tumor tissues according to the general procedure described in Example 1 above. The primary mixture of antibodies comprised three antibodies: (1) anti-ALK monoclonal antibody D5F3®, (2) anti-ROS1 monoclonal antibody D4D6®, and (3) anti-Trk (pan) antibody C17F10 (Cell Signaling Technologies; Beverly, Mass). As shown in FIGS. 3A and 3B, the multiplexed immunohistochemistry assay described herein, when performed on a tumor tissue microarray, showed a wide range of staining intensities, indicating different expression levels of the target biomarkers in tumor tissues.

### Example 3

### Performance of Multiplexed IHC Assays Performed on Tumor Tissue Samples

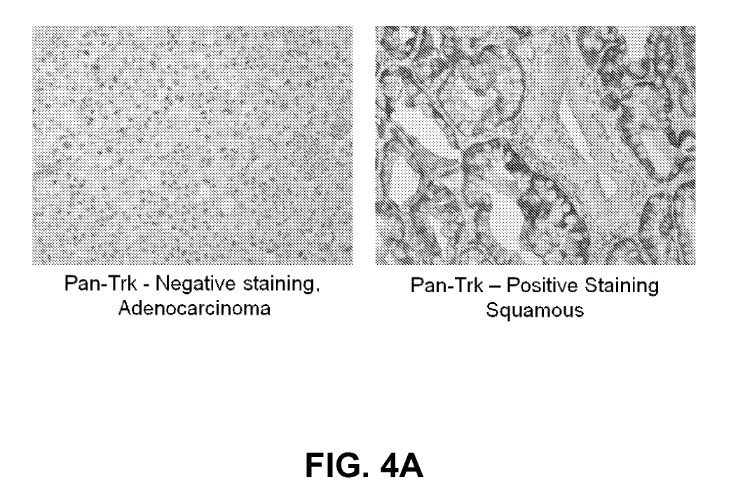

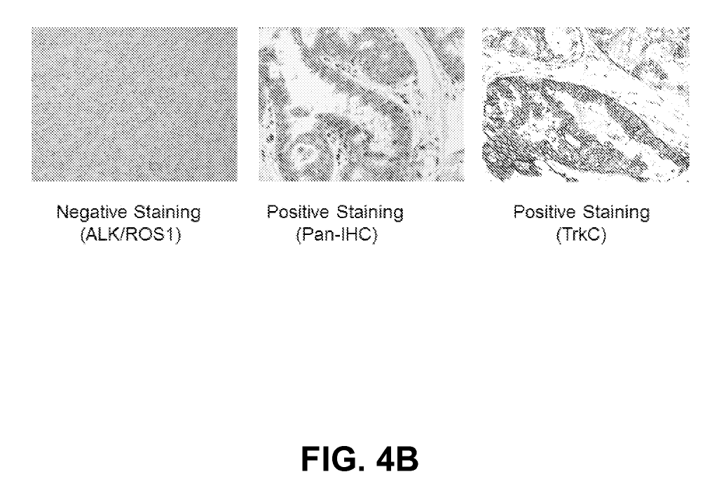

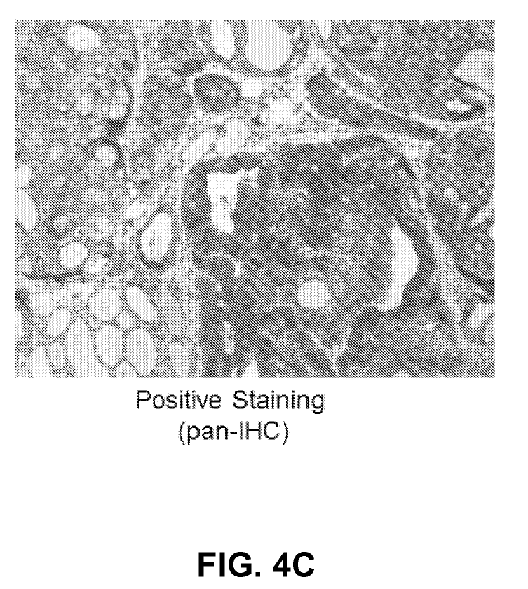

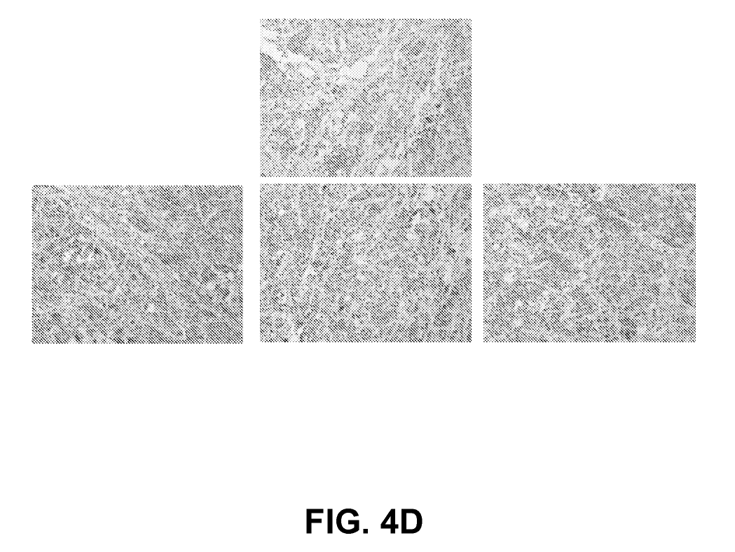

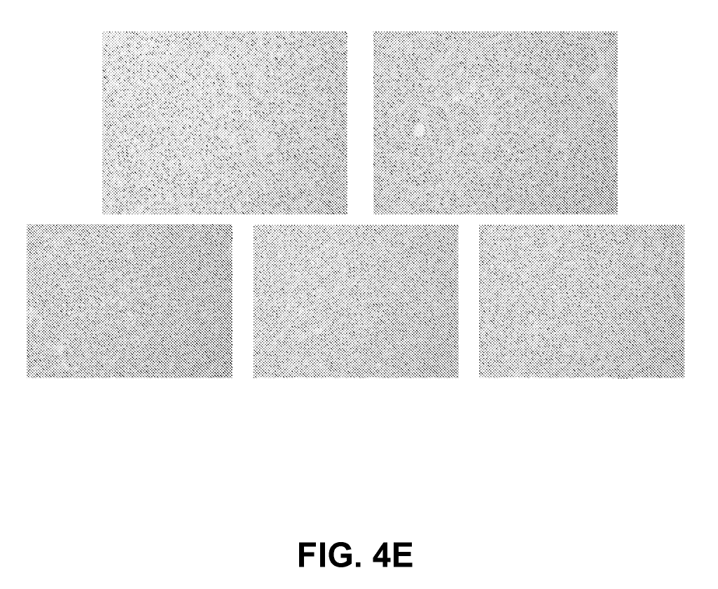

This example describes the use of multiplexed immunohistochemistry assays described herein on various tumor tissue samples in accordance to the general procedure described in Example 1 above. The results were summarized in FIGS. 4A to 4E.

FIG. 4A depicts differential expression of Trk in squamous lung carcinoma and adenocarcinoma, as determined by an IHC assay using the mixture of antibodies described in Example 1. TrkA and TrkB expression has been reported in the scientific literature for squamous lung carcinoma, positive staining consistent with reports in the scientific literature was observed (right panel). In sharp contrast, adenocarcinoma is typically negative for ALK, ROS1, and pan-Trk, and therefore negative staining was observed (left panel).

FIG. 4B illustrates the correlation of positive staining and a gene arrangement, ETV6:NTRK3 gene fusion, which had been previously identified to be present in secretory breast cancer cells. As expected, positive staining was observed with the mixture of three antibodies described in Example 1 (middle panel) and anti-TrkC antibody (right panel), but negative staining was observed when the sample was individually stained for ALK and ROS1 (left panel).

FIG. 4C illustrates the correlation of positive staining and a gene arrangement of the NTRK3 biomarker. The tissue sample was a papillary thyroid cancer sample which had been previously identified to possess ETV6:NTRK3 gene fusion. As expected, positive staining was observed with the mixture of the three antibodies described in Example 1.

FIG. 4D illustrates the correlation of negative staining and absence of background staining in colorectal cancer cells. Tumor tissues were stained with a mixture of antibodies as described in Example 1 (top panel) and individual antibodies TrkA, TrkB, and TrkC (bottom panels).

FIG. 4E illustrates the correlation of negative staining and absence of background staining in anaplastic large cell lymphoma (ALCL). Tissues were stained with antibodies specific to ALK and ROS1 (top left panel), a mixture of antibodies to each of ALK, ROS1, TrkA, TrkB, and TrkC (top right panel), and antibodies specific to each of TrkA, TrkB and TrkC (bottom row).

Taken together, the experimental results presented in Examples 1-3 demonstrate that relevant one or more molecular alterations may be detected in a biological sample derived from a patient using an assay that comprises one or more antibodies that binds to one or more of ALK, ROS1, TrkA, TrkB and TrkC.

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

1. A method for treating cancer in a patient, comprising:
a) acquiring knowledge of the presence of one or more molecular alterations in a biological sample from said cancer patient, wherein said one or more molecular alterations is detected by an assay comprising one or more antibodies that bind to one or more of ALK, ROS1, TrkA, TrkB, and TrkC biomarkers;
b) selecting a chemotherapeutic agent as a treatment for said cancer patient wherein said assay detects the presence of one or more of said one or more molecular alterations, and wherein said selected chemotherapeutic agent is one or more of N-[5-(3,5-difluorobenzyl)-1H-indazol-3-yl]-4-(piperazin-1-yl)-2-(tetrahydro-2H-pyran-4-ylamino) benzamide, N-[5-(3,5-difluorobenzyl)-1H-indazol-3-yl]-4-(4-methyl-piperazin-1-yl)-2-(tetrahydro-2H-pyran-4-ylamino) benzamide, and N-[5-(3,5-difluoro-benzenesulfonyl)-1H-indazol-3-yl]-2-((R)-2-methoxy-1-methyl-ethylamino)-4-(4-methyl-piperazin-1-yl) benzamide, or a pharmaceutically acceptable salt thereof; and
c) administering a therapeutically effective amount of said one or more selected chemotherapeutic agents to said cancer patient.

2. The method of claim 1, wherein said selected chemotherapeutic agent is N-[5-(3,5-difluorobenzyl)-1H-indazol-3-yl]-4-(piperazin-1-yl)-2-(tetrahydro-2H-pyran-4-ylamino) benzamide, or a pharmaceutically acceptable salt thereof.

3. The method of claim 1, wherein said selected chemotherapeutic agent is N-[5-(3,5-difluorobenzyl)-1H-indazol-3-yl]-4-(4-methyl-piperazin-1-yl)-2-(tetrahydro-2H-pyran-4-ylamino) benzamide, or a pharmaceutically acceptable salt thereof.

4. The method of claim 1, wherein said selected chemotherapeutic agent is N-[5-(3,5-difluoro-benzenesulfonyl)-1H-indazol-3-yl]-2-((R)-2-methoxy-1-methyl-ethylamino)-4-(4-methyl-piperazin-1-yl) benzamide, or a pharmaceutically acceptable thereof.

5. (canceled)

6. The method of claim 1, wherein said one or more molecular alterations detected in said biological sample involve at least two, at least three, or at least four of said biomarkers.

7. The method of claim 1, where said knowledge is acquired from:
a) an assay that comprises contacting said biological sample simultaneously or sequentially with one or more antibodies or fragments thereof specific for said biomarkers, wherein said specific antibodies are optionally monoclonal antibodies or said specific antibodies optionally comprise at least one of D5F3®, D4D5®, C17F1 , and combinations thereof; or
b) an antibody-based assay selected form the group consisting of ELISA, immunohistochemistry, western blotting, mass spectrometry, flow cytometry, protein-microarray, immunofluorescence, and a multiplex detection assay; or
c) an assay performed simultaneously on a plurality of biological samples, wherein said plurality of biological samples optionally comprises at least 6, 12, 24, 48, 96, 200, 384, 400, 500, 1000, 1500, or 3000 samples.

8-11. (canceled)

12. The method of claim 1, wherein said one or more molecular alterations results in elevated expression of one or more of ALK, ROS1, TrkA, TrkB, and TrkC biomarkers.

13. The method of claim 12, wherein the knowledge of said one or more molecular alterations is acquired from an assay wherein determining whether the expression of one or more biomarker is elevated comprises:
a) determining the expression level of said one or more biomarkers in said biological sample; and
b) comparing said determined expression level to a reference expression level.

14-15. (canceled)

16. The method of claim 1, further comprising acquiring knowledge of a genetic alteration in the cancer of said patient from a second analytical assay prior to the administering step, wherein said second analytical assay is selected from the group consisting of capillary electrophoresis, nucleic acid sequencing, polypeptide sequencing, restriction digestion, nucleic acid amplification-based assays, nucleic acid hybridization assay, comparative genomic hybridization, real-time PCR, quantitative reverse transcription PCR (qRT-PCR), PCR-RFLP assay, HPLC, mass-spectrometric genotyping, fluorescent in-situ hybridization (FISH), next generation sequencing (NGS), and a kinase activity assay.

17. The method of claim 1, wherein said cancer is selected from the group consisting of anaplastic large-cell lymphoma (ALCL), colorectal cancer (CRC), cholangiocarcinoma, gastric, glioblastomas (GBM), leiomyosarcoma, melanoma, non-small cell lung cancer (NSCLC), squamous cell lung cancer, neuroblastoma (NB), ovarian cancer, pancreatic cancer, prostate cancer, medullary thyroid cancer, breast cancer, and papillary thyroid cancer.

18-19. (canceled)

20. The method of claim 1, wherein said one or more molecular alterations is selected from a genetic mutation, a gene amplification, a gene rearrangement, a single-nucleotide variation (SNV), a deletion, an insertion, an InDel mutation, a single nucleotide point mutation (SNP), an epigenetic alteration, a splicing variant, an RNA/protein overexpression, and an aberrant RNA/protein expression, and any combination thereof.

21. The method of claim 20, wherein said one or more molecular alterations comprises an insertion of a heterologous nucleic acid sequence within a coding sequence of a biomarker gene, and wherein said insertion optionally forms a chimeric nucleic acid sequence that encodes a fusion peptide.

22. (canceled)

23. The method of claim 1, wherein acquiring knowledge of said one or more molecular alterations further comprises determining a nucleic acid sequence and/or an amino acid sequence comprising said one or more molecular alterations.

24. The method of claim 1, wherein said selected chemotherapeutic agent or a pharmaceutically acceptable salt thereof is administered as a single therapeutic agent or in combination with a second therapeutic agent.

25. The method of claim 1, said method comprising administering to said patient a therapeutically effective amount of said selected chemotherapeutic agent, or a pharmaceutically accepted salt thereof, in multiple dosages for a treatment period of 2 to 50 days or a treatment period of 5-42 days.

26. (canceled)

27. The method of claim 25, said selected chemotherapeutic agent or a pharmaceutically acceptable salt thereof is administered to said patient with an oral dosage of about 60 mg/kg twice a day (BID), seven times per week or with an oral dosage of about 60 mg/kg twice a day (BID), seven times per week for six weeks, on alternate weekly basis.

28. (canceled)

29. A method for selecting a cancer patient who is predicted to respond to the administration of a therapeutic regimen, comprising:
a) acquiring knowledge of the presence of one or more molecular alterations in a biological sample from said cancer patient, wherein said one or more molecular alterations is detected by an assay comprising one or more antibodies that bind to one or more of ALK, ROS1, TrkA, TrkB, and TrkC biomarkers; and
b) selecting the patient as predicted to respond to the administration of a therapeutic regimen if said one or more molecular alterations is detected in one or more of said biomarkers; or
selecting the patient as predicted to not respond to the administration of a therapeutic regimen if said one or more molecular alterations is not detected in said biomarkers,
wherein said therapeutic regiment comprises administering to said selected patient a therapeutically effective amount of one or more chemotherapeutic agents selected from N-[5-(3,5-difluorobenzyl)-1H-indazol-3-yl]-4-(piperazin-1-yl)-2-(tetrahydro-2H-pyran-4-ylamino) benzamide, N-[5-(3,5-difluorobenzyl)-1H-indazol-3-yl]-4-(4-methyl-piperazin-1-yl)-2-(tetrahydro-2H-pyran-4-ylamino) benzamide, and N-[5-(3,5-difluoro-benzenesulfonyl)-1H-indazol-3-yl]-2-((R)-2-methoxy-1-methyl-ethylamino)-4-(4-methyl-piperazin-1-yl) benzamide, or a pharmaceutically acceptable salt thereof.

30. The method of claim 29, wherein said selected chemotherapeutic agent is N-[5-(3,5-difluorobenzyl)-1H-indazol-3-yl]-4-(piperazin-1-yl)-2-(tetrahydro-2H-pyran-4-yl amino) benzamide, or a pharmaceutically acceptable salt thereof.

31. The method of claim 29, wherein said selected chemotherapeutic agent is N-[5-(3,5-difluorobenzyl)-1H-indazol-3-yl]-4-(4-methyl-piperazin-1-yl)-2-(tetrahydro-2H-pyran-4-ylamino) benzamide, or a pharmaceutically acceptable salt thereof.

32. The method of claim 29, wherein said selected chemotherapeutic agent is N-[5-(3,5-difluoro-benzenesulfonyl)-1H-indazol-3-yl]-2-((R)-2-methoxy-1-methyl-ethylamino)-4-(4-methyl-piperazin-1-yl) benzamide, or a pharmaceutically acceptable thereof.
